SEQUENCE LISTING (28 sequences)

SEQ ID NO: 1 - protein, 514 aa, Artificial
  chimeric antibody huHEA125, heavy chain, membrane-bound form
EVKLLESGGGLVQPGGSLKLSCAASGFDFSRFWMTWVRQAPGKGLEWIGEINLDSSTINYTPSLKDKFIISRDNAKNTLFLQMSKVRSEDTALYYCSRGISMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGLQLDETCAEAQDGELDGLWTTITIFISLFLLSVCYSAAVTLFKVKWIFSSVVELKQTLVPEYKNMIGQAP

SEQ ID NO: 2 - protein, 445 aa, Artificial
  chimeric antibody huHEA125, heavy chain, secreted form
EVKLLESGGGLVQPGGSLKLSCAASGFDFSRFWMTWVRQAPGKGLEWIGEINLDSSTINYTPSLKDKFIISRDNAKNTLFLQMSKVRSEDTALYYCSRGISMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 115 aa, Artificial
  chimeric antibody huHEA125, heavy chain, VH domain
EVKLLESGGGLVQPGGSLKLSCAASGFDFSRFWMTWVRQAPGKGLEWIGEINLDSSTINYTPSLKDKFIISRDNAKNTLFLQMSKVRSEDTALYYCSRGISMDYWGQGTSVTVSS

SEQ ID NO: 4 - DNA, 75 bp, Artificial
  chimeric antibody huHEA125, heavy chain, FR1 segment
gaagtgaagcttctcgagtctggaggtggcctggtgcagcctggaggatccctgaaactctcctgtgcagcctca

SEQ ID NO: 5 - DNA, 24 bp, Artificial
  chimeric antibody huHEA125, heavy chain, CDR1 segment
ggattcgattttagtagattctgg

SEQ ID NO: 6 - DNA, 51 bp, Artificial
  chimeric antibody huHEA125, heavy chain, FR2 segment
atgacttgggtccggcaggctccagggaaagggctagaatggattggagaa

SEQ ID NO: 7 - DNA, 24 bp, Artificial
  chimeric antibody huHEA125, heavy chain, CDR2 segment
attaatctagatagcagtacgata

SEQ ID NO: 8 - DNA, 114 bp, Artificial
  chimeric antibody huHEA125, heavy chain, FR3 segment
aactatacgccatctctaaaggataaattcatcatctccagggacaacgccaaaaatacgctgttcctgcaaatgagcaaagtgagatctgaggacacagccctttattactgt

SEQ ID NO: 9 - DNA, 24 bp, Artificial
  chimeric antibody huHEA125, heavy chain, CDR3 segment
tcaagaggtatttctatggactac

SEQ ID NO: 10 - DNA, 33 bp, Artificial
  chimeric antibody huHEA125, heavy chain, FR4 segment
tggggtcagggaacctcagtcaccgtctcctca

SEQ ID NO: 11 - protein, 214 aa, Artificial
  chimeric antibody huHEA125, light chain
DILLTQSPAILSVSPGERVSFSCRASQSIGISLHWYQQRPSDSPRLLIKYASESISGIPSRFSGSGSGTDFTLSINSVESEDIADYYCQQSNIWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 107 aa, Artificial
  chimeric antibody huHEA125, light chain, VL domain
DILLTQSPAILSVSPGERVSFSCRASQSIGISLHWYQQRPSDSPRLLIKYASESISGIPSRFSGSGSGTDFTLSINSVESEDIADYYCQQSNIWPTTFGAGTKLELK

SEQ ID NO: 13 - DNA, 78 bp, Artificial
  chimeric antibody huHEA125, light chain, FR1 segment
gacatcttgctgactcagtctccagccatcctgtctgtgagtccaggagaaagagtcagtttctcctgcagggccagt

SEQ ID NO: 14 - DNA, 18 bp, Artificial
  chimeric antibody huHEA125, light chain, CDR1 segment
cagagcattggcataagt

SEQ ID NO: 15 - DNA, 51 bp, Artificial
  chimeric antibody huHEA125, light chain, FR2 segment
ttacactggtatcagcaaagaccaagtgattctccaaggcttctcataaag

SEQ ID NO: 16 - DNA, 9 bp, Artificial
  chimeric antibody huHEA125, light chain, CDR2 segment
tatgcttct

SEQ ID NO: 17 - DNA, 108 bp, Artificial
  chimeric antibody huHEA125, light chain, FR3 segment
gagtcaatctctgggatcccttccaggtttagtggcagtggatcagggacagattttactcttagcatcaacagtgtggagtctgaagatattgcagattattactgt

SEQ ID NO: 18 - DNA, 27 bp, Artificial
  chimeric antibody huHEA125, light chain, CDR3 segment
caacaaagtaatatctggccaaccacg

SEQ ID NO: 19 - DNA, 30 bp, Artificial
  chimeric antibody huHEA125, light chain, FR4 segment
ttcggtgctgggaccaagctggagctgaaa

SEQ ID NO: 20 - protein, 8 aa, Artificial
  chimeric antibody huHEA125, heavy chain, CDR1 domain
GFDFSRFW

SEQ ID NO: 21 - protein, 8 aa, Artificial
  chimeric antibody huHEA125, heavy chain, CDR2 domain
INLDSSTI

SEQ ID NO: 22 - protein, 8 aa, Artificial
  chimeric antibody huHEA125, heavy chain, CDR3 domain
SRGISMDY

SEQ ID NO: 23 - protein, 6 aa, Artificial
  chimeric antibody huHEA125, light chain, CDR1 domain
QSIGIS

SEQ ID NO: 24 - protein, 3 aa, Artificial
  chimeric antibody huHEA125, light chain, CDR2 domain
YAS

SEQ ID NO: 25 - protein, 9 aa, Artificial
  chimeric antibody huHEA125, light chain, CDR3 domain
QQSNIWPTT

SEQ ID NO: 26 - protein, 399 aa, Artificial
  chimeric antibody huHEA125, heavy chain, constant domain, membrane-bound form
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGLQLDETCAEAQDGELDGLWTTITIFISLFLLSVCYSAAVTLFKVKWIFSSVVELKQTLVPEYKNMIGQAP

SEQ ID NO: 27 - protein, 330 aa, Artificial [sequence omitted]

SEQ ID NO: 28 - protein, 107 aa, Artificial [sequence omitted]

(19) United States
(12) Patent Application Publication
(10) Pub. No.: US 2012/0213805 A1
(43) Pub. Date: Aug. 23, 2012

(54) Amatoxin-Armed Tartget-Binding Moieties for the Treatment of Cancer

(76) Inventors: Heinz Faulstich, Heidelberg (DE); Frank Breitling, Heidelberg (DE); Sandra Lüttgau, Bad Schonborn (DE); Gerhard Moldenhauer, Heidelberg (DE)

(21) Appl. No.: 13/260,328

(22) PCT Filed: Apr. 8, 2010
(86) PCT No.: PCT/EP2010/002206

§ 371 Date: Jan. 16, 2012

Related U.S. Application Data
(60) Provisional application No. 61/167,690, filed on Apr. 8, 2009.

(51) Int. Cl. A61K 39/395; A61P 35/00; A61P 35/02; C07K 19/00

(52) U.S. Cl. 424/183.1; 530/387.3

(57) ABSTRACT

The invention relates to tumour therapy. In one aspect, the present invention relates to conjugates of target-binding moieties and toxins that are useful in the treatment of cancer. In particular, the toxin is an amatoxin, and the target-binding moieties (e.g. antibodies) are directed against tumour-associated antigens, such as epithelial cell adhesion molecule (EpCAM). In a further aspect the invention relates to pharmaceutical compositions comprising such target-binding moiety toxin conjugates and to the use of such target-binding moiety toxin conjugates for the preparation of such pharmaceutical compositions. The target-binding moiety toxin conjugates and pharmaceutical compositions of the invention are useful for the treatment of cancer, in particular adenocarcinoma, such as pancreatic cancer, cholangiocarcinoma, breast cancer, and colorectal cancer.

### FIELD OF THE INVENTION

The invention relates to tumour therapy. In one aspect, the present invention relates to conjugates of target-binding moieties and toxins that are useful in the treatment of cancer. In particular, the toxin is an amatoxin, and the target-binding moieties (e.g. antibodies) are directed against tumour-associated antigens, such as epithelial cell adhesion molecule (EpCAM). In a further aspect the invention relates to pharmaceutical compositions comprising such target-binding moiety toxin conjugates and to the use of such target-binding moiety toxin conjugates for the preparation of such pharmaceutical compositions. The target-binding moiety toxin conjugates and pharmaceutical compositions of the invention are useful for the treatment of cancer, in particular adenocarcinoma, such as pancreatic cancer, cholangiocarcinoma, breast cancer, and colorectal cancer.

### BACKGROUND OF THE INVENTION AND STATE OF THE ART

### Amatoxins

Amatoxins are cyclic peptides composed of 8 amino acids. They can be isolated from Amanita phalloides mushrooms or prepared from the building blocks by synthesis. Amatoxins specifically inhibit the DNA-dependent RNA polymerase II of mammalian cells, and thereby also the transcription and protein biosynthesis of the affected cells. Inhibition of transcription in a cell causes stop of growth and proliferation. Though not covalently bound, the complex between amanitin and RNA-polymerase II is very tight (K_{D}=3 nM). Dissociation of amanitin from the enzyme is a very slow process what makes recovery of an affected cell unlikely. When the inhibition of transcription lasts too long, the cell will undergo programmed cell death (apoptosis).

### Epithelial Cell Adhesion Molecule

Epithelial cell adhesion molecule (EpCAM, CD326) is one of the best-studied target antigens on human tumors (Trzpis et al., 2007; Baeuerle and Gires, 2007). It represents a type I membrane glycoprotein of 314 amino acids with an apparent molecular weight of 40 kDa (Balzar et al., 1999). It is overexpressed in the majority of adenocarcinomas (Winter et al., 2003; Went et al., 2004). In particular, EpCAM expression is enhanced in node-positive breast cancer, epithelial ovarian cancer, cholangiocarcinoma, pancreatic adenocarcinoma and squamous cell head and neck cancer. Increased EpCAM expression is indicative for a poor prognosis in breast and gallbladder carcinomas (Gastl et al., 2000; Varga et al., 2004; Spizzo et al., 2002; Spizzo et al., 2004). Importantly, EpCAM is expressed by tumor initiating or cancer stem cells in mammary, colorectal and pancreatic carcinomas (Al-Hajj et al., 2003; Dalerba et al., 2007; Li et al., 2007).

EpCAM-specific monoclonal antibodies have been used as a diagnostic tool for the detection of rare circulating tumor cells in cancer patients (Allard et al., 2004; Nagrath et al., 2007). A couple of engineered anti-EpCAM antibodies are currently investigated in clinical studies.

### Conjugates of Amatoxins and Antibodies

Earlier patent application EP 1 859 811 A1 (published Nov. 28, 2007) by the inventors describes conjugates, in which β-amanitin is coupled to albumin or to the monoclonal antibodies HEA125, OKT3, and PA-1. Furthermore, the inhibitory effect of these conjugates on the proliferation of breast cancer cells (MCF-7), Burkitt's lymphoma cells (Raji), and T-lymphoma cells (Jurkat) was studied.

### TECHNICAL PROBLEMS UNDERLYING THE PRESENT INVENTION

There was a need in the prior art for target-binding moiety toxin conjugates that exert their toxic effects to target cells or tissues at much lower concentration. Furthermore, a need remained in the prior art for the treatment of other types of diseases, in particular for the treatment of other types of cancer, particularly those being therapy resistant, or poorly responding to actual tumour therapies.

The present invention fulfils these and other needs. For example, the inventors found out in the experiments underlying the present invention that conjugates comprising amatoxins and the new chimeric antibody huHEA125 are capable of inhibiting tumour cell proliferation at much lower concentrations than the conjugates described in the prior art. In particular, conjugates comprising amatoxins and the chimeric antibody huHEA125 exert their inhibitory effect at a concentration that is about one hundredth of the concentration needed when using conjugates of the prior art. Furthermore, the inventors discovered that conjugates comprising amatoxins and EpCAM-specific antibodies cannot only inhibit proliferation of breast cancer cells but are surprisingly also capable of inhibiting proliferation of pancreatic adenocarcinoma cells, colorectal cancer cells, and cholangiocarcinoma cells. Additionally, the inventors found out that choosing a particular linkage point in the amatoxin part of the conjugates yields highly effective target-binding moiety toxin conjugates (in particular antibody toxin conjugates) that exert their toxic activity on the target cells at very low concentrations (IC₅₀ around 2×10⁻¹² to 2×10⁻¹¹ M) and that are highly specific for their target cells. Without wishing to be bound by a particular theory, this latter advantage might be explained in that the amatoxin is efficiently released from the target-binding moiety amatoxin conjugate inside the target cell but not outside the cell.

The above overview does not necessarily describe all problems solved by the present invention.

### SUMMARY OF THE INVENTION

In a first aspect the present invention relates to an antibody toxin conjugate for the treatment of pancreatic cancer, cholangiocarcinoma, or colorectal cancer in a patient, wherein the conjugate comprises (i) an antibody or antigen binding fragment thereof specifically binding to an epitope of epithelial cell adhesion molecule (EpCAM); (ii) an amatoxin; and (iii) optionally a linker L1.

In a second aspect the present invention relates to an antibody toxin conjugate comprising (i) an antibody or an antigen binding fragment thereof specifically binding to epithelial cell adhesion molecule (EpCAM), wherein the antibody or an antigen binding fragment thereof comprises: (a) the heavy chain of huHEA125, wherein the heavy chain is selected from the group consisting of: (a1) the membrane-bound form of the heavy chain according to SEQ ID NO: 1, wherein the variable domain of the heavy chain VH as shown in SEQ ID NO: 3 comprises between 0 and 10 amino acid exchanges, between 0 and 10 amino acid deletions and/or between 0 and 10 amino acid additions positioned in the framework regions of VH, and wherein the constant domain of the heavy chain as shown in SEQ ID NO: 26 comprises between 0 and 10 amino acid exchanges, between 0 and 10 amino acid deletions and/or between 0 and 10 amino acid additions; and (a2) the soluble form of the heavy chain according to SEQ ID NO: 2, wherein the variable domain of the heavy chain VH as shown in SEQ ID NO: 3 comprises between 0 and 10 amino acid exchanges, between 0 and 10 amino acid deletions and/or between 0 and 10 amino acid additions positioned in the framework regions of VH, and wherein the constant domain of the heavy chain as shown in SEQ ID NO: 27 comprises between 0 and 10 amino acid exchanges, between 0 and 10 amino acid deletions and/or between 0 and 10 amino acid additions; and (b) the light chain of huHEA125 according to SEQ ID NO: 11, wherein the variable domain of the light chain VL as shown in SEQ ID NO: 12 comprises between 0 and 10 amino acid exchanges, between 0 and 10 amino acid deletions and/or between 0 and 10 amino acid additions positioned in the framework regions of VL, and wherein the constant domain of the light chain CL as shown in SEQ ID NO: 28 comprises between 0 and 10 amino acid exchanges, between 0 and 10 amino acid deletions and/or between 0 and 10 amino acid additions; (ii) an amatoxin; and (iii) optionally a linker L2.

In a third aspect the present invention relates to an antibody toxin conjugate according to the second aspect for use in medicine.

In a fourth aspect the present invention relates to an antibody toxin conjugate according to the second aspect for the treatment of cancer in a patient, wherein the cancer is selected from the group consisting of pancreatic cancer, cholangiocarcinoma, breast cancer and colon cancer.

In a fifth aspect the present invention relates to a target-binding moiety toxin conjugate comprising: (i) a target-binding moiety; (ii) an amatoxin; and (iii) optionally a linker L3; wherein the amatoxin is connected to the target-binding moiety or, if present, to the linker L3 via the δ C-atom of amatoxin amino acid 3.

In an sixth aspect the present invention relates to a target-binding moiety toxin conjugate according to the fifth aspect for use in medicine.

In a seventh aspect the present invention relates to a target-binding moiety toxin conjugate according to the fifth aspect for the treatment of cancer in a patient, wherein the cancer is selected from the group consisting of pancreatic cancer, cholangiocarcinoma, breast cancer, colorectal cancer, lung cancer, prostate cancer, ovarian cancer, stomach cancer, kidney cancer, malignant melanoma, leukemia and malignant lymphoma.

In an eighth aspect the present invention relates to a pharmaceutical composition comprising the antibody toxin conjugate according to the first aspect or the second aspect or the target-binding moiety toxin conjugate according to the fifth aspect and further comprising one or more pharmaceutically acceptable diluents, carriers, excipients, fillers, binders, lubricants, glidants, disintegrants, adsorbents; and/or preservatives.

This summary of the invention does not necessarily describe all features of the invention.

### BRIEF DESCRIPTION OF THE DRAWINGS

FIG. 1 shows the structural formulae of different amatoxins. The numbers in bold type (1 to 8) designate the standard numbering of the eight amino acids forming the amatoxin. The most important carbon atoms in amino acid 3 are labelled with Greek letter α, β, γ, and δ. The atom numbers in the side chain of the (substituted) tryptophan, i.e. amino acid no. 4, are also shown (numbers 1′ to 7′).

FIG. 2 shows a comparison of the binding affinities of huHEA125-Ama and huHEA125 to target cells by a binding competition analysis. EpCAM-expressing Colo205 cells were incubated with a fixed amount of directly FITC-labeled mouse HEA125 antibody. Binding to target cells was analyzed by flow cytometry. Competition of binding with increasing amounts of huHEA125-Ama or huHEA125 revealed a very similar affinity towards the target antigen.

FIG. 3 shows the surface expression of EpCAM antigen on various carcinoma cell lines detected by indirect immunofluorescence: FIG. 3A Capan-1 (human pancreatic adenocarcinoma); FIG. 3B Colo205 (human colon adenocarcinoma); FIG. 3C OZ (human cholangiocarcinoma); and FIG. 3D MCF-7 (human breast adenocarcinoma line), FIG. 3E BxPC-3 (human pancreatic adenocarcinoma); and FIG. 3F PC-3 (human prostate adenocarcinoma). The grey-shaded histograms on the left side of each diagram show the results obtained with control antibody Xolair®; the histograms having a white area on the right side of each diagram show the results obtained with antibody huHEA125.

FIG. 4 shows a comparison of the inhibition of Capan-1 cell proliferation caused by Amanitin-armed antibody huHEA125, Amanitin-armed control antibody Xolair®, and free Amanitin.

FIG. 5 shows a comparison of the inhibition of Colo205 cell proliferation caused by Amanitin-armed antibody huHEA125, Amanitin-armed control antibody Xolair®, and free Amanitin.

FIG. 6 shows a comparison of the inhibition of MCF-7 cell proliferation caused by Amanitin-armed antibody huHEA125, Amanitin-armed control antibody Xolair®, and free Amanitin.

FIG. 7 shows a comparison of the inhibition of OZ cell proliferation caused by Amanitin-armed antibody huHEA125, Amanitin-armed control antibody Xolair®, and free Amanitin.

FIG. 8 shows the inhibition of BxPC-3 cell proliferation caused by Amanitin-armed antibody huHEA125, and Amanitin-armed control antibody Xolair® and free Amanitin for comparison.

FIG. 9 shows growth inhibition of BxPC-3 tumor xenografts in NOD/SCID mice after huHEA125-amanitin treatment.

FIG. 10 shows growth inhibition of PC-3 tumor xenografts in NOD/SCID mice after huHEA125-amanitin treatment.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention.

The term “target-binding moiety”, as used herein, refers to any molecule or part of a molecule that can specifically bind to a target molecule or target epitope. Preferred target-binding moieties in the context of the present application are (i) antibodies or antigen-binding fragments thereof; (ii) antibody-like proteins; and (iii) nucleic acid aptamers. “Target-binding moieties” suitable for use in the present invention typically have a molecular mass of 40 000 Da (40 kDa) or more.

In the context of the present application the terms “target molecule” and “target epitope”, respectively, refers to an antigen and an epitope of an antigen, respectively, that is specifically bound by a target-binding moiety, preferably the target molecule is a tumour-associated antigen, in particular an antigen or an epitope, which is present on the surface of one or more tumour cell types in an increased concentration and/or in a different steric configuration as compared to the surface of non-tumour cells. Preferably, said antigen or epitope is present on the surface of one or more tumour cell types but not on the surface of non-tumour cells.

The term “antibody or antigen binding fragment thereof”, as used herein, refers to immunoglobulin molecules and immunologically active portions of immunoglobulin molecules, i.e. molecules that contain an antigen binding site that immunospecifically binds an antigen. Also comprised are immunoglobulin-like proteins that are selected through techniques including, for example, phage display to specifically bind to a target molecule, e.g. to the target protein EpCAM. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. “Antibodies and antigen-binding fragments thereof” suitable for use in the present invention include, but are not limited to, polyclonal, monoclonal, monovalent, bispecific, heteroconjugate, multispecific, human, humanized (in particular CDR-grafted), deimmunized, or chimeric antibodies, single chain antibodies (e.g. scFv), Fab fragments, F(ab′)₂ fragments, fragments produced by a Fab expression library, diabodies or tetrabodies (Holliger P. et al., 1993), nanobodies, anti-idiotypic (anti-Id) antibodies (including, e.g., anti-Id antibodies to antibodies of the invention), and epitope-binding fragments of any of the above.

In some embodiments the antigen-binding fragments are human antigen-binding antibody fragments of the present invention and include, but are not limited to, Fab, Fab′ and F(ab′)₂, Fd, single-chain Fvs (scFv), single-chain antibodies, disulfide-linked Fvs (dsFv) and fragments comprising either a VL or VH domain. Antigen-binding antibody fragments, including single-chain antibodies, may comprise the variable domain(s) alone or in combination with the entirety or a portion of the following: hinge region, CL, CH1, CH2, and CH3 domains. Also included in the invention are antigen-binding fragments also comprising any combination of variable domain(s) with a hinge region, CL, CH1, CH2, and CH3 domains.

Antibodies usable in the invention may be from any animal origin including birds and mammals. Preferably, the antibodies are human, rodent (e.g. mouse and rat), donkey, sheep rabbit, goat, guinea pig, camel, horse, or chicken. It is particularly preferred that the antibodies are of human or murine origin. As used herein, “human antibodies” include antibodies having the amino acid sequence of a human immunoglobulin and include antibodies isolated from human immunoglobulin libraries or from animals transgenic for one or more human immunoglobulin and that do not express endogenous immunoglobulins, as described for example in U.S. Pat. No. 5,939,598 by Kucherlapati & Jakobovits.

The term “antibody-like protein” refers to a protein that has been engineered (e.g. by mutagenesis of loops) to specifically bind to a target molecule. Typically, such an antibody-like protein comprises at least one variable peptide loop attached at both ends to a protein scaffold. This double structural constraint greatly increases the binding affinity of the antibody-like protein to levels comparable to that of an antibody. The length of the variable peptide loop typically consists of 1.0 to 20 amino acids. The scaffold protein may be any protein having good solubility properties. Preferably, the scaffold protein is a small globular protein. Antibody-like proteins include without limitation affibodies, anticalins, and designed ankyrin repeat proteins (for review see: Binz et al. 2005). Antibody-like proteins can be derived from large libraries of mutants, e.g. be panned from large phage display libraries and can be isolated in analogy to regular antibodies. Also, antibody-like binding proteins can be obtained by combinatorial mutagenesis of surface-exposed residues in globular proteins.

The term “nucleic acid aptamer” refers to a nucleic acid molecule that has been engineered through repeated rounds of in vitro selection or SELEX (systematic evolution of ligands by exponential enrichment) to bind to a target molecule (for a review see: Brody and Gold, 2000). The nucleic acid aptamer may be a DNA or RNA molecule. The aptamers may contain modifications, e.g. modified nucleotides such as 2′-fluorine-substituted pyrimidines.

The term “amatoxin” includes all cyclic peptides composed of 8 amino acids as isolated from the genus Amanita and described in ref. (Wieland, T. and Faulstich H., 1978); further all chemical derivatives thereof; further all semisynthetic analogs thereof; further all synthetic analogs thereof built from building blocks according to the master structure of the natural compounds (cyclic, 8 aminoacids), further all synthetic or semisynthetic analogs containing non-hydroxylated amino acids instead of the hydroxylated amino acids, further all synthetic or semisynthetic analogs, in which the thioether sulfoxide moiety is replaced by a sulfide, sulfone, or by atoms different from sulfur, e.g. a carbon atom as in a carbaanalog of amanitin.

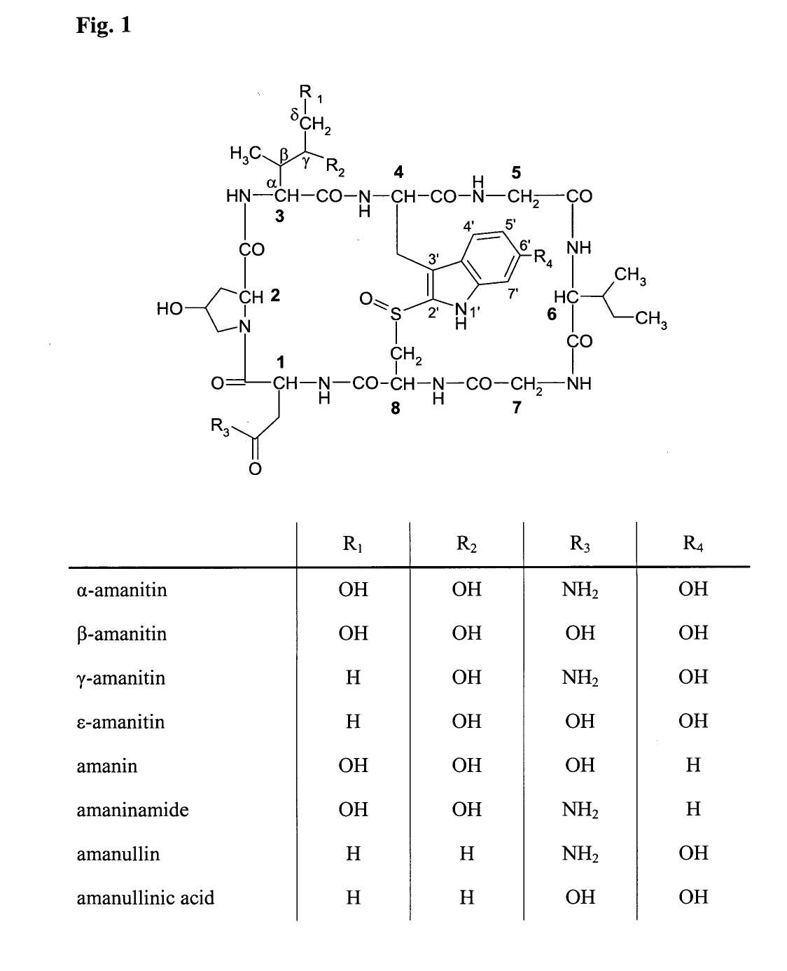

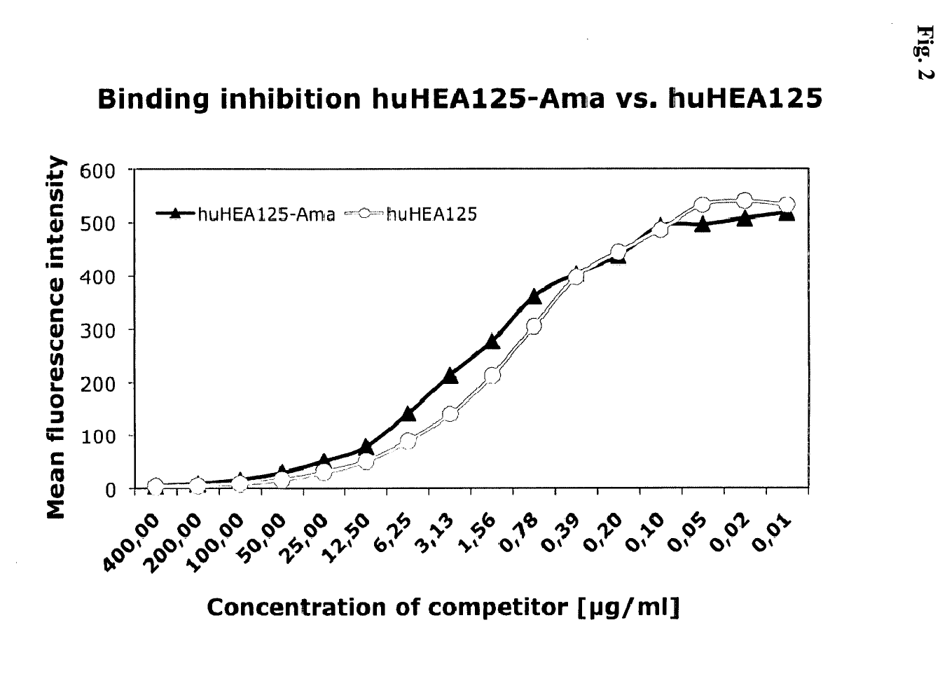

Functionally, amatoxins are defined as peptides or depsipeptides that inhibit mammalian RNA polymerase II. Preferred amatoxins are those with a functional group (e.g. a carboxylic group, an amino group, a hydroxy group, a thiol or a thiol-capturing group) that can be reacted with linker molecules or proteins, such as antibodies or antibody fragments. Amatoxins which are particularly suitable for the conjugates of the present invention are α-amanitin, β-amanitin, γ-amanitin, £-amanitin, amanin, amaninamide, amanullin, and amanullinic acid as shown in FIG. 1 as well as salts, chemical derivatives, semisynthetic analogs, and synthetic analogs thereof. Particularly preferred amatoxins for use in the present invention are α-amanitin, β-amanitin, and amaninamide.

As used herein, a “derivative” of a compound refers to a species having a chemical structure that is similar to the compound, yet containing at least one chemical group not present in the compound and/or deficient of at least one chemical group that is present in the compound. The compound to which the derivative is compared is known as the “parent” compound. Typically, a “derivative” may be produced from the parent compound in one or more chemical reaction steps.

As used herein, an “analog” of a compound is structurally related but not identical to the compound and exhibits at least one activity of the compound. The compound to which the analog is compared is known as the “parent” compound. The afore-mentioned activities include, without limitation: binding activity to another compound; inhibitory activity, e.g. enzyme inhibitory activity; toxic effects; activating activity, e.g. enzyme-activating activity. It is not required that the analog exhibits such an activity to the same extent as the parent compound. A compound is regarded as an analog within the context of the present application, if it exhibits the relevant activity to a degree of at least 1% (more preferably at least 5%, more preferably at least 10%, more preferably at least 20%, more preferably at least 30%, more preferably at least 40%, and more preferably at least 50%) of the activity of the parent compound. Thus, an “analog of an amatoxin”, as it is used herein, refers to a compound that is structurally related to any one of α-amanitin, β-amanitin, γ-amanitin, £-amanitin, amanin, amaninamide, amanullin, and amanullinic acid as shown in FIG. 1 and that exhibits at least 1% (more preferably at least 5%, more preferably at least 10%, more preferably at least 20%, more preferably at least 30%, more preferably at least 40%, and more preferably at least 50%) of the inhibitory activity against mammalian RNA polymerase II as compared to at least one of α-amanitin, β-amanitin, γ-amanitin, £-amanitin, amanin, amaninamide, amanullin, and amanullinic acid. An “analog of an amatoxin” suitable for use in the present invention may even exhibit a greater inhibitory activity against mammalian RNA polymerase II than any one of α-amanitin, β-amanitin, γ-amanitin, £-amanitin, amanin, amaninamide, amanullin, or amanullinic acid. The inhibitory activity might be measured by determining the concentration at which 50% inhibition occurs (IC₅₀ value).

A “linker” in the context of the present application refers to a molecule that increases the distance between two components, e.g. to alleviate steric interference between the target-binding moiety and the amatoxin, which may otherwise decrease the ability of the amatoxin to interact with RNA polymerase II. The linker may serve another purpose as it may facilitate the release of the amatoxin specifically in the cell being targeted by the target binding moiety. It is preferred that the linker and preferably the bond between the linker and the amatoxin on one side and the bond between the linker and the antibody on the other side is stable under the physiological conditions outside the cell, e.g. the blood, while it can be cleaved inside the cell, in particular inside the target cell, e.g. cancer cell or immune cell. To provide this selective stability the linker may comprise functionalities that are preferably pH-sensitive to generate pH-sensitive linkers as described, e.g. in S. Fletcher, M. R. Jorgensens and A. D. Miller; Org. Lett. 2004, 6(23), pp 4245-4248, or protease sensitive to generate protease sensitive linkers as described, e.g. in L. D A Ibsen, Blood 2003, 102, 1458-65 or Francisco J A, Cerreny C G, Meyer D L, Nat. Biotechnol 2003, 21, 778-84. Alternatively, the bond linking the linker to the target binding moiety may provide the selective stability. Preferably a linker has a length of at least 1, preferably of 1-20 atoms length (e.g. 1, 2, 3, 4, 5, 6, 7, 8, 9, 10, 11, 12, 13, 14, 15, 16, 17, 18, 19, or 20 atoms) wherein one side of the linker has been reacted with the amatoxin and, the other side with a target-binding moiety. In the context of the present invention, a linker preferably is a C₁₋₂₀-alkyl, C₁₋₂₀-heteroalkyl, C₂₋₂₀-alkenyl, C₂₋₂₀-heteroalkenyl, C₂₋₂₀-alkynyl, C₂₋₂₀-heteroalkynyl, cycloalkyl, heterocycloalkyl, aryl, heteroaryl, aralkyl, or a heteroaralkyl group, optionally substituted. The linker may contain one or more structural elements such as amide, ester, ether, thioether, disulfide, hydrocarbon moieties and the like. The linker may also contain combinations of two or more of these structural elements. Each one of these structural elements may be present in the linker more than once, e.g. twice, three times, four times, five times, or six times. In some embodiments the linker may comprise a disulfide bond. It is understood that the linker has to be attached either in a single step or in two or more subsequent steps to the amatoxin and the target binding moiety. To that end the linker to be will carry two groups, preferably at a proximal and distal end, which can (i) form a covalent bond to a group, preferably an activated group on an amatoxin or the target binding-peptide or (ii) which is or can be activated to form a covalent bond with a group on an amatoxin. Accordingly, if the linker is present, it is preferred that chemical groups are at the distal and proximal end of the linker, which are the result of such a coupling reaction, e.g. an ester, an ether, a urethane, a peptide bond etc. The presence of a “linker” is optional, i.e. the toxin may be directly linked to a residue of the target-binding moiety in some embodiments of the target-binding moiety toxin conjugate of the present invention. It is preferred that the linker is connected directly via a bond to the targeting moiety, preferably at its terminus. If the target-binding moiety comprises free amino, carboxy or sulfhydryl groups, e.g. in the form of Asp, Glu, Arg, Lys, Cys residues, which may be comprised in a polypeptide, than it is preferred that the linker is coupled to such a group.

As used herein, a first compound (e.g. an antibody) is considered to “specifically bind” to a second compound (e.g. an antigen, such as a target protein), if it has a dissociation constant K_{D} to said second compound of 100 μM or less, preferably 50 μM or less, preferably 30 μM or less, preferably 20 μM or less, preferably 10 μM or less, preferably 5 μM or less, more preferably 1 μM or less, more preferably 900 nM or less, more preferably 800 nM or less, more preferably 700 nM or less, more preferably 600 nM or less, more preferably 500 nM or less, more preferably 400 nM or less, more preferably 300 nM or less, more preferably 200 nM or less, even more preferably 100 nM or less, even more preferably 90 nM or less, even more preferably 80 nM or less, even more preferably 70 nM or less, even more preferably 60 nM or less, even more preferably 50 nM or less, even more preferably 40 nM or less, even more preferably 30 nM or less, even more preferably 20 nM or less, and even more preferably 10 nM or less.

As used herein, a “patient” means any mammal or bird who may benefit from a treatment with the target-binding moiety toxin conjugates described herein. Preferably, a “patient” is selected from the group consisting of laboratory animals (e.g. mouse or rat), domestic animals (including e.g. guinea pig, rabbit, donkey, sheep, goat, chicken, camel, horse, cat, or dog), or primates including human beings. It is particularly preferred that the “patient” is a human being.

As used herein, “treat”, “treating” or “treatment” of a disease or disorder means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, “administering” includes in vivo administration, as well as administration directly to tissue ex vivo, such as vein grafts.

An “effective amount” is an amount of a therapeutic agent sufficient to achieve the intended purpose. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

“Pharmaceutically acceptable” means approved by a regulatory agency of the Federal or a state government or listed in the U.S. Pharmacopeia or other generally recognized pharmacopeia for use in animals, and more particularly in humans.

### Embodiments of the Invention

The present invention will now be further described. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In a first aspect the present invention is directed to an antibody toxin conjugate for the treatment of pancreatic cancer, cholangiocarcinoma, or colorectal cancer in a patient, wherein the conjugate comprises (i) an antibody or antigen binding fragment thereof specifically binding to an epitope of epithelial cell adhesion molecule (EpCAM); (ii) an amatoxin; and (iii) optionally a linker.

In a preferred embodiment of the first aspect the antibody or antigen binding fragment thereof is selected from a diabody, a tetrabody, a nanobody, a chimeric antibody, a deimmunized antibody, a humanized antibody or a human antibody. In a preferred embodiment of the first aspect the antigen binding fragment is selected from the group consisting of Fab, F(ab′)₂, Fd, Fv, single-chain Fv, and disulfide-linked Fvs (dsFv).

In a preferred embodiment the epitope of EpCAM is an epitope of human EpCAM. In a preferred embodiment of the first aspect the antibody or the antigen binding fragment thereof comprises (a) the CDR3 domain (SEQ ID NO: 22) of the heavy chain of huHEA125; and/or (b) the CDR3 domain (SEQ ID NO: 25) of the light chain of huHEA125. In a particularly preferred embodiment, the antibody or the antigen binding fragment thereof comprises both of these CDR3 domains as set forth in SEQ ID NO: 22 and SEQ ID NO: 25. Preferably, the antibody or the antigen binding fragment thereof additionally comprises one or more of the following: (a) the CDR2 domain (SEQ ID NO: 21) of the heavy chain of huHEA125; (b) the CDR1 domain (SEQ ID NO: 20) of the heavy chain of huHEA125; (c) the CDR2 domain (SEQ ID NO: 24) of the light chain of huHEA125; and (d) the CDR1 domain (SEQ ID NO: 23) of the light chain of huHEA125. In a preferred embodiment the antibody or the antigen binding fragment thereof comprises the CDR3 domain (SEQ ID NO: 22), the CDR2 domain (SEQ ID NO: 21), and the CDR1 domain (SEQ ID NO: 20) of the heavy chain of huHEA125. In a preferred embodiment the antibody or the antigen binding fragment thereof comprises the CDR3 domain (SEQ ID NO: 25), the CDR2 domain (SEQ ID NO: 24), and the CDR1 domain (SEQ ID NO: 23) of the light chain of huHEA125. In a particularly preferred embodiment, the antibody or the antigen binding fragment thereof comprises the CDR3 domains, the CDR2 domains, and the CDR1 domains of the heavy chain and the light chain, i.e. the antibody or the antigen binding fragment thereof comprises the amino acid sequences as set forth in SEQ ID NO: 20, 21, 22, 23, 24, and 25.

In a preferred embodiment of the first aspect the antibody or the antigen binding fragment thereof comprises the variable domain of the heavy chain (=VH) of huHEA125 (SEQ ID NO: 3) and/or variable domain of the light chain (=VL) of huHEA125 (SEQ ID NO: 12). In a particularly preferred embodiment, the antibody or the antigen binding fragment thereof comprises both the VH domain (SEQ ID NO: 3) and the VL domain (SEQ ID NO: 12) of huHEA125.

In a preferred embodiment of the first aspect the antibody or the antigen binding fragment thereof comprises the heavy chain of huHEA125 (soluble form, SEQ ID NO: 2) and/or the light chain of huHEA125 (SEQ ID NO: 11). In one embodiment, the heavy chain of huHEA125 and/or the light chain of huHEA125 each comprise independently from each other up to 20 (e.g. 1, 2, 3, 4, 5, 6, 7, 8, 9, 10, 11, 12, 13, 14, 15, 16, 17, 18, 19 or 20) amino acid exchanges, deletions, or additions, wherein these amino acid exchanges, deletions, or additions may be positioned in the constant domains of the heavy chain and/or in the constant domain of the light chain and/or in the framework regions of the variable domain of the heavy chain and/or in the framework regions of the variable domain of the light chain. In a particularly preferred embodiment, the antibody is a complete IgG antibody comprising two heavy chains of huHEA125 (SEQ ID NO: 2) and two light chains of huHEA125 (SEQ ID NO: 11), wherein one heavy chain is connected to one light chain via a disulfide linkage and wherein the heavy chains are connected to each other by one or two (preferably two) disulfide linkages.

In a preferred embodiment of the first aspect the antibody or the antigen binding fragment thereof comprises the heavy chain of huHEA125 (membrane-bound form, SEQ ID NO: 1) and/or the light chain of huHEA125 (SEQ ID NO: 11). In one embodiment, the heavy chain of huHEA125 and/or the light chain of huHEA125 each comprise independently from each other up to 20 (e.g. 1, 2, 3, 4, 5, 6, 7, 8, 9, 10, 11, 12, 13, 14, 15, 16, 17, 18, 19 or 20) amino acid exchanges, deletions, or additions, wherein these amino acid exchanges, deletions, or additions may be positioned in the constant domains of the heavy chain and/or in the constant domain of the light chain and/or in the framework regions of the variable domain of the heavy chain and/or in the framework regions of the variable domain of the light chain. In a particularly preferred embodiment, the antibody is a complete IgG antibody comprising two heavy chains of huHEA125 (SEQ ID NO: 1) and two light chains of huHEA125 (SEQ ID NO: 11), wherein one heavy chain is connected to one light chain via a disulfide linkage and wherein the heavy chains are connected to each other by one or two (preferably two) disulfide linkages.

In a preferred embodiment of the first aspect the amatoxin is selected from α-amanitin, β-amanitin, γ-amanitin, £-amanitin, amanin, amaninamide, amanullin, and amanullinic acid (all shown in FIG. 1), as well as salts, chemical derivatives, semisynthetic analogs, and synthetic analogs thereof. Particularly preferred amatoxins are α-amanitin, β-amanitin, and amaninamide, as well as salts, chemical derivatives, semisynthetic analogs, and synthetic analogs thereof. In a preferred embodiment of the first aspect the amatoxin is connected to the antibody or, if present, to the linker L1 via the δC-atom of amatoxin amino acid 3 (see FIG. 1). In preferred amatoxins usable in the present invention said amino acid 3 is isoleucine, γ-hydroxy-isoleucine or γ,δ-dihydroxy-isoleucine.

In preferred embodiments of the first aspect, the amatoxin is connected to the antibody or, if present, to the linker L1 via an oxygen atom bound to the δC-atom of amatoxin amino acid 3. It is further preferred that the amatoxin is connected to the antibody or, if present, to the linker L1 via an ester linkage, an ether linkage or a urethane linkage. In these embodiments, it is preferred that amino acid 3 is γ,δ-dihydroxy-isoleucine.

In preferred embodiments of the first aspect, the antibody is connected to the amatoxin or, if present, to the linker L1 via an amino group present in the antibody.

In a preferred embodiment of the first aspect the linker L1 is an alkyl, heteroalkyl, alkenyl, heteroalkenyl, alkynyl, heteroalkynyl, cycloalkyl, heterocycloalkyl, aryl, heteroaryl, aralkyl, or a heteroaralkyl group, optionally substituted. In further preferred embodiments of the first aspect the linker L1 comprises a disulfide bond.

In a second aspect the present invention is directed to an antibody toxin conjugate comprising (i) an antibody or an antigen binding fragment thereof specifically binding to epithelial cell adhesion molecule (EpCAM), wherein the antibody or an antigen binding fragment thereof comprises: (a) the heavy chain of huHEA125, wherein the heavy chain is selected from the group consisting of: (a1) the membrane-bound form of the heavy chain according to SEQ ID NO: 1, wherein the variable domain of the heavy chain VH as shown in SEQ ID NO: 3 comprises between 0 and 10 (e.g. 0, 1, 2, 3, 4, 5, 6, 7, 8, 9, or 10) amino acid exchanges, between 0 and 10 (e.g. 0, 1, 2, 3, 4, 5, 6, 7, 8, 9, or 10) amino acid deletions and/or between 0 and 10 (e.g. 0, 1, 2, 3, 4, 5, 6, 7, 8, 9, or 10) amino acid additions positioned in the framework regions of VH, and wherein the constant domain of the heavy chain as shown in SEQ ID NO: 26 comprises between 0 and 10 (e.g. 0, 1, 2, 3, 4, 5, 6, 7, 8, 9, or 10) amino acid exchanges, between 0 and 10 (e.g. 0, 1, 2, 3, 4, 5, 6, 7, 8, 9, or 10) amino acid deletions and/or between 0 and 10 (e.g. 0, 1, 2, 3, 4, 5, 6, 7, 8, 9, or 10) amino acid additions; and (a2) the soluble form of the heavy chain according to SEQ ID NO: 2, wherein the variable domain of the heavy chain VH as shown in SEQ ID NO: 3 comprises between 0 and 10 (e.g. 0, 1, 2, 3, 4, 5, 6, 7, 8, 9, or 10) amino acid exchanges, between 0 and 10 (e.g. 0, 1, 2, 3, 4, 5, 6, 7, 8, 9, or 10) amino acid deletions and/or between 0 and 10 (e.g. 0, 1, 2, 3, 4, 5, 6, 7, 8, 9, or 10) amino acid additions positioned in the framework regions of VH, and wherein the constant domain of the heavy chain as shown in SEQ ID NO: 27 comprises between 0 and 10 (e.g. 0, 1, 2, 3, 4, 5, 6, 7, 8, 9, or 10) amino acid exchanges, between 0 and 10 (e.g. 0, 1, 2, 3, 4, 5, 6, 7, 8, 9, or 10) amino acid deletions and/or between 0 and 10 (e.g. 0, 1, 2, 3, 4, 5, 6, 7, 8, 9, or 10) amino acid additions; and (b) the light chain of huHEA125 according to SEQ ID NO: 11, wherein the variable domain of the light chain VL as shown in SEQ ID NO: 12 comprises between 0 and 10 (e.g. 0, 1, 2, 3, 4, 5, 6, 7, 8, 9, or 10) amino acid exchanges, between 0 and 10 (e.g. 0, 1, 2, 3, 4, 5, 6, 7, 8, 9, or 10) amino acid deletions and/or between 0 and 10 (e.g. 0, 1, 2, 3, 4, 5, 6, 7, 8, 9, or 10) amino acid additions positioned in the framework regions of VL, and wherein the constant domain of the light chain CL as shown in SEQ ID NO: 28 comprises between 0 and 10 (e.g. 0, 1, 2, 3, 4, 5, 6, 7, 8, 9, or 10) amino acid exchanges, between 0 and 10 (e.g. 0, 1, 2, 3, 4, 5, 6, 7, 8, 9, or 10) amino acid deletions and/or between 0 and 10 (e.g. 0, 1, 2, 3, 4, 5, 6, 7, 8, 9, or 10) amino acid additions; (ii) an amatoxin; and (iii) optionally a linker.

In a preferred embodiment of the second aspect the antibody or an antigen binding fragment thereof comprises: (a) the heavy chain of huHEA125, wherein the heavy chain is selected from the group consisting of: (a1) the membrane-bound form of the heavy chain according to SEQ ID NO: 1, wherein the variable domain of the heavy chain VH as shown in SEQ ID NO: 3 comprises between 0 and 10 (e.g. 0, 1, 2, 3, 4, 5, 6, 7, 8, 9, or 10) amino acid exchanges, between 0 and 10 (e.g. 0, 1, 2, 3, 4, 5, 6, 7, 8, 9, or 10) amino acid deletions and/or between 0 and 10 (e.g. 0, 1, 2, 3, 4, 5, 6, 7, 8, 9, or 10) amino acid additions positioned in the framework regions of VH; and (a2) the soluble form of the heavy chain according to SEQ ID NO: 2, wherein the variable domain of the heavy chain VH as shown in SEQ ID NO: 3 comprises between 0 and 10 (e.g. 0, 1, 2, 3, 4, 5, 6, 7, 8, 9, or 10) amino acid exchanges, between 0 and 10 (e.g. 0, 1, 2, 3, 4, 5, 6, 7, 8, 9, or 10) amino acid deletions and/or between 0 and 10 (e.g. 0, 1, 2, 3, 4, 5, 6, 7, 8, 9, or 10) amino acid additions positioned in the framework regions of VH; and (b) the light chain of huHEA125 according to SEQ ID NO: 11, wherein the variable domain of the light chain VL as shown in SEQ ID NO: 12 comprises between 0 and 10 (e.g. 0, 1, 2, 3, 4, 5, 6, 7, 8, 9, or 10) amino acid exchanges, between 0 and 10 (e.g. 0, 1, 2, 3, 4, 5, 6, 7, 8, 9, or 10) amino acid deletions and/or between 0 and 10 (e.g. 0, 1, 2, 3, 4, 5, 6, 7, 8, 9, or 10) amino acid additions positioned in the framework regions of VL.

In a preferred embodiment of the second aspect the antibody or an antigen binding fragment thereof comprises: (a) the heavy chain of huHEA125, wherein the heavy chain is selected from the group consisting of: (a1) the membrane-bound form of the heavy chain according to SEQ ID NO: 1, wherein the variable domain of the heavy chain VH as shown in SEQ ID NO: 3 comprises between 0 and 10 (e.g. 0, 1, 2, 3, 4, 5, 6, 7, 8, 9, or 10) amino acid exchanges, amino acid deletions and/or amino acid additions positioned in the framework regions of VH, and wherein the constant domain of the heavy chain as shown in SEQ ID NO: 26 comprises between 0 and 10 (e.g. 0, 1, 2, 3, 4, 5, 6, 7, 8, 9, or 10) amino acid exchanges, amino acid deletions and/or amino acid additions; and (a2) the soluble form of the heavy chain according to SEQ ID NO: 2, wherein the variable domain of the heavy chain VH as shown in SEQ ID NO: 3 comprises between 0 and 10 (e.g. 0, 1, 2, 3, 4, 5, 6, 7, 8, 9, or 10) amino acid exchanges, amino acid deletions and/or amino acid additions positioned in the framework regions of VH, and wherein the constant domain of the heavy chain as shown in SEQ ID NO: 27 comprises between 0 and 10 (e.g. 0, 1, 2, 3, 4, 5, 6, 7, 8, 9, or 10) amino acid exchanges, amino acid deletions and/or amino acid additions; and (b) the light chain of huHEA125 according to SEQ ID NO: 11, wherein the variable domain of the light chain VL as shown in SEQ ID NO: 12 comprises between 0 and 10 (e.g. 0, 1, 2, 3, 4, 5, 6, 7, 8, 9, or 10) amino acid exchanges, amino acid deletions and/or amino acid additions positioned in the framework regions of VL, and wherein the constant domain of the light chain CL as shown in SEQ ID NO: 28 comprises between 0 and 10 (e.g. 0, 1, 2, 3, 4, 5, 6, 7, 8, 9, or 10) amino acid exchanges, amino acid deletions and/or amino acid additions.

In a preferred embodiment of the second aspect the antibody or an antigen binding fragment thereof comprises: (a) the heavy chain of huHEA125, wherein the heavy chain is selected from the group consisting of: (a1) the membrane-bound form of the heavy chain according to SEQ ID NO: 1, wherein the variable domain of the heavy chain VH as shown in SEQ ID NO: 3 comprises between 0 and 10 (e.g. 0, 1, 2, 3, 4, 5, 6, 7, 8, 9, or 10) amino acid exchanges positioned in the framework regions of VH, and wherein the constant domain of the heavy chain as shown in SEQ ID NO: 26 comprises between 0 and 10 (e.g. 0, 1, 2, 3, 4, 5, 6, 7, 8, 9, or 10) amino acid exchanges; and (a2) the soluble form of the heavy chain according to SEQ ID NO: 2, wherein the variable domain of the heavy chain VH as shown in SEQ ID NO: 3 comprises between 0 and 10 (e.g. 0, 1, 2, 3, 4, 5, 6, 7, 8, 9, or 10) amino acid exchanges positioned in the framework regions of VH, and wherein the constant domain of the heavy chain as shown in SEQ ID NO: 27 comprises between 0 and 10 (e.g. 0, 1, 2, 3, 4, 5, 6, 7, 8, 9, or 10) amino acid exchanges; and (b) the light chain of huHEA125 according to SEQ ID NO: 11, wherein the variable domain of the light chain VL as shown in SEQ ID NO: 12 comprises between 0 and 10 (e.g. 0, 1, 2, 3, 4, 5, 6, 7, 8, 9, or 10) amino acid exchanges positioned in the framework regions of VL, and wherein the constant domain of the light chain CL as shown in SEQ ID NO: 28 comprises between 0 and 10 (e.g. 0, 1, 2, 3, 4, 5, 6, 7, 8, 9, or 10) amino acid exchanges.

In a preferred embodiment of the second aspect the antibody or an antigen binding fragment thereof comprises: (a) the heavy chain of huHEA125, wherein the heavy chain is selected from the group consisting of: (a1) the membrane-bound form of the heavy chain according to SEQ ID NO: 1; and (a2) the soluble form of the heavy chain according to SEQ ID NO: 2; and (b) the light chain of huHEA125 according to SEQ ID NO: 11.

In a preferred embodiment of the second aspect the antibody or antigen binding fragment thereof is selected from a chimeric antibody, a deimmunized antibody, a humanized antibody or a human antibody. In a preferred embodiment of the second aspect the antigen binding fragment is selected from the group consisting of Fab, F(ab′)₂, and Fd.

In a preferred embodiment of the second aspect the antibody is huHEA125 or an antigen binding fragment thereof.

In a preferred embodiment of the second aspect the antibody or antigen binding fragment thereof specifically binds to human EpCAM.

In a preferred embodiment of the second aspect the amatoxin is selected from α-amanitin, β-amanitin, γ-amanitin, £-amanitin, amanin, amaninamide, amanullin, and amanullinic acid (all shown in FIG. 1), as well as salts, chemical derivatives, semisynthetic analogs, and synthetic analogs thereof. Particularly preferred amatoxins are α-amanitin, β-amanitin, and amaninamide, as well as salts, chemical derivatives, semisynthetic analogs, and synthetic analogs thereof.

In a preferred embodiment of the second aspect the amatoxin is connected to the antibody or, if present, to the linker L2 via the SC-atom of amatoxin amino acid 3 (see FIG. 1). In preferred amatoxins usable in the present invention said amino acid 3 is isoleucine, γ-hydroxy-isoleucine or γ,δ-dihydroxy-isoleucine.

In preferred embodiments of the second aspect, the amatoxin is connected to the antibody or, if present, to the linker L2 via an oxygen atom bound to the δC-atom of amatoxin amino acid 3. It is further preferred that the amatoxin is connected to the antibody or, if present, to the linker L2 via an ester linkage, an ether linkage or a urethane linkage. In these embodiments, it is preferred that amino acid 3 is γ,δ-dihydroxy-isoleucine.

In preferred embodiments of the second aspect, the antibody is connected to the amatoxin or, if present, to the linker L2 via an amino group present in the antibody.

In a preferred embodiment of the second aspect the linker L2 is an alkyl, heteroalkyl, alkenyl, heteroalkenyl, alkynyl, heteroalkynyl, cycloalkyl, heterocycloalkyl, aryl, heteroaryl, aralkyl, or a heteroaralkyl group, optionally substituted. In further preferred embodiments of the fifth aspect the linker L2 comprises a disulfide bond.

In a third aspect the present invention is directed to the conjugate of the second aspect for use in medicine.

In a fourth aspect the present invention is directed to the conjugate of the second aspect for the treatment of cancer in a patient, wherein the cancer is selected from the group consisting of pancreatic cancer, cholangiocarcinoma, breast cancer and colorectal cancer.

In a fifth aspect the present invention is directed to the conjugate of the second aspect for the preparation of a pharmaceutical composition for the treatment of cancer in a patient, wherein the cancer is selected from the group consisting of pancreatic cancer, cholangiocarcinoma, breast cancer and colorectal cancer.

In a fifth aspect the present invention relates to a target-binding moiety toxin conjugate comprising: (i) a target-binding moiety; (ii) an amatoxin; and (iii) optionally a linker L3; wherein the amatoxin is connected to the target-binding moiety or, if present, to the linker L3 via the amatoxin amino acid 3, preferably the δC-atom of amatoxin amino acid 3 (see FIG. 1). In preferred amatoxins usable in the present invention said amino acid 3 is isoleucine, γ-hydroxy-isoleucine or γ,δ-dihydroxy-isoleucine.

In a preferred embodiment of the fifth aspect the amatoxin is connected to the target-binding moiety or, if present, to the linker L3 via an oxygen atom bound to the δC-atom of amatoxin amino acid 3. It is further preferred that the amatoxin is connected to the target-binding moiety or, if present, to the linker L3 via an ester linkage, preferably in the form of an amatoxin-O—C(O)-L3-target-binding moiety or an amatoxin-O—C(O)-target-binding moiety, more preferably an amatoxin-δC-O—C(O)-L3-target-binding moiety or an amatoxin-δC—O—C(O-target-binding moiety and most preferably an amatoxin-δCH₂-O—C(O)-L3-target-binding moiety or an amatoxin-δCH₂-O—C(O)-target-binding moiety; an ether linkage, preferably in the form of an amatoxin-O-L3 or an amatoxin-O-target binding moiety, preferably an amatoxin-δC-O-L3-target binding moiety or an amatoxin-δC-O-target binding moiety, more preferably an amatoxin-δCH₂-O-L3-target binding moiety or an amatoxin-δCH₂-O-target binding moiety; or an urethane linkage preferably in the form of an amatoxin-O—C(O)—NH-L3-target-binding moiety or an amatoxin-O—C(O)—NH-target-binding moiety, preferably an amatoxin-δC-O—C(O)—NH-L3-target-binding moiety or an amatoxin-δC—O—C(O)—NH-target-binding moiety, i.e. an amatoxin-δCH₂—O—C(O)—NH-L3-target-binding moiety or an amatoxin-δCH₂—O—C(O)—NH-target-binding moiety. In these embodiments, it is preferred that amino acid 3 is γ,δ-dihydroxy-isoleucine.

In preferred embodiments of the fifth aspect the linker L3 is present and the conjugate has one of the following structures: (i) amatoxin-δC—O—C(O)-L3-C(O)—NH-target-binding moiety; (ii) amatoxin-δC—O-L3-C(O)—NH-target-binding moiety; or (iii) amatoxin-δC—O—C(O)—NH-L3-C(O)—NH-target-binding moiety, preferably (i) amatoxin-δCH₂—O—C(O)-L3-C(O)—NH-target-binding moiety; (ii) amatoxin-δCH₂—O-L3-C(O)—NH-target-binding moiety; or (iii) amatoxin-δCH₂—O—C(O)—NH-L3-C(O)—NH-target-binding moiety.

In a preferred embodiment of the fifth aspect the target-binding moiety is connected to the amatoxin or, if present, to the linker L3 via an amino group present in the target-binding moiety.

In a preferred embodiment of the fifth aspect the amatoxin is selected from α-amanitin, β-amanitin, γ-amanitin, £-amanitin, amanin, amaninamide, amanullin, or amanullinic acid (all shown in FIG. 1), as well as salts, chemical derivatives, semisynthetic analogs, and synthetic analogs thereof. Particularly preferred amatoxins are α-amanitin, β-amanitin, and amaninamide, as well as salts, chemical derivatives, semisynthetic analogs, and synthetic analogs thereof.

In a preferred embodiment of the fifth aspect the linker L3 is an alkyl, heteroalkyl, alkenyl, heteroalkenyl, alkynyl, heteroalkynyl, cycloalkyl, heterocycloalkyl, aryl, heteroaryl, aralkyl, or a heteroaralkyl group, optionally substituted. In further preferred embodiments of the fifth aspect the linker L3 comprises a disulfide bond.

In a preferred embodiment of the fifth aspect the target-binding moiety specifically binds to an epitope that is present on a tumour cell. It is particularly preferred that the target-binding moiety specifically binds to an epitope of epithelial cell adhesion molecule (EpCAM).

In a preferred embodiment of the fifth aspect the target binding moiety is selected from the group consisting of: (i) antibody or antigen-binding fragment thereof; (ii) antibody-like protein; and (iii) nucleic acid aptamer. In a preferred embodiment the antibody or the antigen-binding fragment thereof is selected from a diabody, a tetrabody, a nanobody, a chimeric antibody, a deimmunized antibody, a humanized antibody or a human antibody. In a preferred embodiment the antigen binding fragment is selected from the group consisting of Fab, F(ab′)₂, Fd, Fv, single-chain Fv, and disulfide-linked Fvs (dsFv). In a preferred embodiment the antibody or the antigen binding fragment thereof comprises (a) either the membrane-bound form of the heavy chain of huHEA125 (SEQ ID NO: 1) or the soluble form of the heavy chain of huHEA125 (SEQ ID NO: 2); and/or (b) the light chain of huHEA125 (SEQ ID NO: 11).

In an sixth aspect the present invention relates to a target-binding moiety toxin conjugate according to the fifth aspect for use in medicine.

In a seventh aspect the present invention relates to a target-binding moiety toxin conjugate according to the fifth aspect for the treatment of cancer in a patient, wherein the cancer is selected from the group consisting of pancreatic cancer, cholangiocarcinoma, breast cancer, colorectal cancer, lung cancer, prostate cancer, ovarian cancer, stomach cancer, kidney cancer, malignant melanoma, leukemia and malignant lymphoma.

In an eighth aspect the present invention is directed to a pharmaceutical composition comprising the antibody toxin conjugate of the first aspect or of the second aspect or the target-binding moiety toxin conjugate according to the fifth aspect and further comprising one or more pharmaceutically acceptable diluents, carriers, excipients, fillers, binders, lubricants, glidants, disintegrants, adsorbents; and/or preservatives.

The target binding moiety of the fifth to seventh embodiment is in preferred embodiments a protein, in particular an antibody. Proteins and in particular antibodies will comprise several amino acids, which allow the coupling of amatoxins. Preferred amino acids have free amino, hydroxy, or carbonyl-groups, including Lys, Gln, Glu, Asp, Asn, Thr, and Ser. Accordingly, it is possible to couple more than one amatoxin molecules to one protein molecule. An increase of the number of amatoxins per molecule will also increase the toxicity. Accordingly, in a preferred embodiment the ratio of antibody of the first to fourth embodiment and he target binding moiety of the fifth to seventh embodiment to amatoxin is between 1 protein molecule to between 1 and 15 amatoxin molecules, preferably 1, 2, 3, 4, 5, 6, 7, 8, 9, 10, 11, 12, 13, 14, or 15. For the purpose of the calculation of the ratio in case of dimmers like IgGs the dimmer is considered as one molecule. Similar ratios are preferred, if the target binding moiety is not a protein.

It is particularly preferred that the pharmaceutical composition of the eighth aspect can be used in the form of systemically administered medicaments. These include parenterals, which comprise among others injectables and infusions. Injectables are formulated either in the form of ampoules or as so called ready-for-use injectables, e.g. ready-to-use syringes or single-use syringes and aside from this in puncturable flasks for multiple withdrawal. The administration of injectables can be in the form of subcutaneous (s.c.), intramuscular (i.m.), intravenous (i.v.) or intracutaneous (i.c.) application. In particular, it is possible to produce the respectively suitable injection formulations as a suspension of crystals, solutions, nanoparticular or a colloid dispersed systems like, e.g. hydrosols.

Injectable formulations can further be produced as concentrates, which can be dissolved or dispersed with aqueous isotonic diluents. The infusion can also be prepared in form of isotonic solutions, fatty emulsions, liposomal formulations and micro-emulsions.

Similar to injectables, infusion formulations can also be prepared in the form of concentrates for dilution. Injectable formulations can also be applied in the form of permanent infusions both in in-patient and ambulant therapy, e.g. by way of mini-pumps.

It is possible to add to parenteral drug formulations, for example, albumin, plasma, expander, surface-active substances, organic diluents, pH-influencing substances, complexing substances or polymeric substances, in particular as substances to influence the adsorption of the target-binding moiety toxin conjugates of the invention to proteins or polymers or they can also be added with the aim to reduce the adsorption of the target-binding moiety toxin conjugates of the invention to materials like injection instruments or packaging-materials, for example, plastic or glass.

The target-binding moiety toxin conjugates of the invention can be bound to microcarriers or nanoparticles in parenterals like, for example, to finely dispersed particles based on poly(meth)acrylates, polylactates, polyglycolates, polyamino acids or polyether urethanes. Parenteral formulations can also be modified as depot preparations, e.g. based on the “multiple unit principle”, if the target-binding moiety toxin conjugates of the invention are introduced in finely dispersed, dispersed and suspended form, respectively, or as a suspension of crystals in the medicament or based on the “single unit principle” if the target-binding moiety toxin conjugate of the invention is enclosed in a formulation, e.g. in a tablet or a rod which is subsequently implanted. These implants or depot medicaments in single unit and multiple unit formulations often consist out of so called biodegradable polymers like e.g. polyesters of lactic and glycolic acid, polyether urethanes, polyamino acids, poly(meth)acrylates or polysaccharides.

Adjuvants and carriers added during the production of the pharmaceutical compositions of the present invention formulated as parenterals are preferably aqua sterilisata (sterilized water), pH value influencing substances like, e.g. organic or inorganic acids or bases as well as salts thereof, buffering substances for adjusting pH values, substances for isotonization like e.g. sodium chloride, sodium hydrogen carbonate, glucose and fructose, tensides and surfactants, respectively, and emulsifiers like, e.g. partial esters of fatty acids of polyoxyethylene sorbitans (for example, Tween®) or, e.g. fatty acid esters of polyoxyethylenes (for example, Cremophor), fatty oils like, e.g. peanut oil, soybean oil or castor oil, synthetic esters of fatty acids like, e.g. ethyl oleate, isopropyl myristate and neutral oil (for example, Miglyol®) as well as polymeric adjuvants like, e.g. gelatine, dextran, polyvinylpyrrolidone, additives which increase the solubility of organic solvents like, e.g. propylene glycol, ethanol, N,N-dimethylacetamide, propylene glycol or complex forming substances like, e.g. citrate and urea, preservatives like, e.g. benzoic acid hydroxypropyl ester and methyl ester, benzyl alcohol, antioxidants like e.g. sodium sulfite and stabilizers like e.g. EDTA.

When formulating the pharmaceutical compositions of the present invention as suspensions in a preferred embodiment thickening agents to prevent the setting of the target-binding moiety toxin conjugates of the invention or, tensides and polyelectrolytes to assure the resuspendability of sediments and/or complex forming agents like, for example, EDTA are added. It is also possible to achieve complexes of the active ingredient with various polymers. Examples of such polymers are polyethylene glycol, polystyrol, carboxymethyl cellulose, Pluronics® or polyethylene glycol sorbit fatty acid ester. The target-binding moiety toxin conjugates of the invention can also be incorporated in liquid formulations in the form of inclusion compounds e.g. with cyclodextrins. In particular embodiments dispersing agents can be added as further adjuvants. For the production of lyophilisates scaffolding agents like mannite, dextran, saccharose, human albumin, lactose, PVP or varieties of gelatine can be used.

In a further aspect the present invention is directed to a method of treating pancreatic cancer, cholangiocarcinoma, or colorectal cancer in a patient in need thereof, comprising administering to the patient an effective amount of an antibody toxin conjugate as defined in the first aspect.

In a further aspect the present invention is directed to a method of treating pancreatic cancer, cholangiocarcinoma, breast cancer or colorectal cancer in a patient in need thereof, comprising administering to the patient an effective amount of an antibody toxin conjugate as defined in the third aspect. In a further aspect the present invention is directed to a method of treating pancreatic cancer, cholangiocarcinoma, breast cancer or colorectal cancer in a patient in need thereof, comprising administering to the patient an effective amount of an target-binding moiety toxin conjugate as defined in the fifth aspect.

### EXAMPLES

In the following, the invention is explained in more detail by non-limiting examples:

### Example 1

### Comparison of Binding Affinities to Target Cells Between Antibody huHEA125 and Antibody Toxin Conjugate amanitin-huHEA125

1.1 Chimeric Antibody huHEA125

Several years ago, the inventors have established a hybridoma cell line secreting the anti-EpCAM mouse monoclonal antibody HEA125 (Moldenhauer et al., 1987; Momburg et al., 1987). Using molecular biology techniques this hybridoma line was reconstructed to produce a chimeric version of the antibody consisting of the mouse variable domains hooked up to human kappa constant light chain and human IgG1 constant heavy chain. The resulting antibody huHEA125 binds to EpCAM-expressing cells with high affinity (K_{d}=2.2×10⁻⁹ M) and high specificity. The gene sequence and the amino acid sequence of huHEA125 immunoglobulin are shown below:

huHEA125 Heavy Chain

Peptide sequence heavy chain, membrane bound form (IGHV/IGHD/IGHJ/IGHG1; IGHG1 is underlined) (SEQ ID NO: 1):

EVKLLESGGGLVQPGGSLKLSCAASGFDFSRFWMTWVRQAPGKGLEWIG
EINLDSSTINYTPSLKDKFIISRDNAKNTLFLQMSKVRSEDTALYYCSR
GISMDYWGQGTSVTVSSASTKGPSVFPLAPSSKSTSGGTAALGCLVKDY
FPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSL
SPGLQLDETCAEAQDGELDGLWTTITIFISLFLLSVCYSAAVTLFKVKW
IFSSVVELKQTLVPEYKNMIGQAP

Peptide sequence heavy chain, secreted form (SEQ ID NO: 2):

EVKLLESGGGLVQPGGSLKLSCAASGFDFSRFWMTWVRQAPGKGLEWIG
EINLDSSTINYTPSLKDKFIISRDNAKNTLFLQMSKVRSEDTALYYCSR
GISMDYWGQGTSVTVSSASTKGPSVFPLAPSSKSTSGGTAALGCLVKDY
FPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSL
SPGK

Peptide sequence (IGHV/IGHD/IGHJ=VH domain; the framework regions FR1, FR2, FR3 and FR4 are underlined) (SEQ ID NO: 3):

EVKLLESGGGLVQPGGSLKLSCAASGFDFSRFWMTWVRQAPGKGLEWIG
EINLDSSTINYTPSLKDKFIISRDNAKNTLFLQMSKVRSEDTALYYCSR
GISMDYWGQGTSVTVSS

Nucleic acid sequence (annotated according to the IMGT-nomenclature, IGHV/IGHD/IGHJ; IGHD underlined; IGHJ doubly underlined):

FR1 (SEQ ID NO: 4):
GAAGTGAAGCTTCTCGAGTCTGGAGGTGGCCTGGTGCAGCCTGGAGGAT
CCCTGAAACTCTCCTGTGCAGCCTCA
CDR1 (SEQ ID NO: 5):
GGATTCGATTTTAGTAGATTCTGG
FR2 (SEQ ID NO: 6):
ATGACTTGGGTCCGGCAGGCTCCAGGGAAAGGGCTAGAATGGATTGGAG
AA
CDR2 (SEQ ID NO: 7):
ATTAATCTAGATAGCAGTACGATA
FR3 (SEQ ID NO: 8):
AACTATACGCCATCTCTAAAGGATAAATTCATCATCTCCAGGGACAACG
CCAAAAATACGCTGTTCCTGCAAATGAGCAAAGTGAGATCTGAGGACAC
AGCCCTTTATTACTGT
CDR3 (SEQ ID NO: 9):
TCAAGAGGTATTTCTATGGACTAC
FR4 (SEQ ID NO: 10):
TGGGGTCAGGGAACCTCAGTCACCGTCTCCTCA

huHEA125 Light Chain

Peptide sequence light chain (IGKV/IGKJ/IGKC; IGKC is underlined) (SEQ ID NO: 11):

DILLTQSPAILSVSPGERVSFSCRASQSIGISLHWYQQRPSDSPRLLIK
YASESISGIPSRFSGSGSGTDFTLSINSVESEDIADYYCQQSNIWPTTF
GAGTKLELKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

Peptide sequence (IGKV/IGKJ=VL domain; the framework regions FR1, FR2, FR3 and FR4 are underlined) (SEQ ID NO: 12):

DILLTQSPAILSVSPGERVSFSCRASQSIGISLHWYQQRPSDSPRLLIK
YASESISGIPSRFSGSGSGTDFTLSINSVESEDIADYYCQQSNIWPTTF
GAGTKLELK

Nucleic acid sequence (annotated according to the IMGT-nomenclature, IGKV/IGKJ; IGKV is underlined; IGKJ is doubly underlined):

FR1 (SEQ ID NO: 13):
GACATCTTGCTGACTCAGTCTCCAGCCATCCTGTCTGTGAGTCCAGGAG
AAAGAGTCAGTTTCTCCTGCAGGGCCAGT
CDR1 (SEQ ID NO: 14):
CAGAGCATTGGCATAAGT
FR2 (SEQ ID NO: 15):
TTACACTGGTATCAGCAAAGACCAAGTGATTCTCCAAGGCTTCTCATAA
AG
CDR2 (SEQ ID NO: 16):
TATGCTTCT
FR3 (SEQ ID NO: 17):
GAGTCAATCTCTGGGATCCCTTCCAGGTTTAGTGGCAGTGGATCAGGGA
CAGATTTTACTCTTAGCATCAACAGTGTGGAGTCTGAAGATATTGCAGA
TTATTACTGT
CDR3 (SEQ ID NO: 18:
CAACAAAGTAATATCTGGCCAACCACG
FR4 (SEQ ID NO: 19):
TTCGGTGCTGGGACCAAGCTGGAGCTGAAA

### 1.2 Control Antibody Xolair®

The control antibody Xolair® (Omalizumab, human IgG1 antibody directed against human IgE immunoglobulin) was produced by Novartis, Germany.

### 1.3 Synthesis of α-Amanitin Antibody Conjugate

### 1.3.1 Synthesis of α-Amanitin-glutarate

3.0 mg (3.3 μmol) of α-amanitin, dried in vacuo over P₄O₁₀ was dissolved in 0.25 ml of dry pyridine and reacted with 0.9 mg (79 μmol) glutaric anhydride in 0.1 ml pyridine for 24 h at RT in the dark. The peptide was precipitated by addition of 7 ml of dry diethylether, centrifuged, and the solid washed a second time with diethylether and centrifuged.

By way of this reaction an α-amanitin derivative is obtained wherein R₁=—OH (in FIG. 1) is replaced by R₁=—O—C(O)—(CH₂)₃—COOH.

1.3.2 Synthesis of α-Amanitin-glutaric acid N-hydroxysuccinimidate

3.4 mg of α-amanitin glutarate (3.3 μmol) was dissolved in 0.05 ml of dry dimethylformamide (DMF), and 2.4 mg (7 eq.) of N-hydroxy-succinimide dissolved in 0.01 ml of DMF were added. After the addition of 1.2 mg of dicyclohexylcarbodiimide in 0.01 ml of DMF the reaction was allowed to proceed for 16 h at RT. The solution was separated from the crystals formed, and the peptide precipitated by the addition of 4 ml of dry diethylether. After centrifugation, the pellet was washed with another 4 ml of ether and centrifuged. The solid was dissolved in 0.1 ml of dimethylformamide and immediately used for the reaction with the antibody solution.

### 1.3.3 Synthesis of α-Amanitin-glutarate-huHEA125

0.1 ml of the solution of 3.0 mg of α-amanitin-glutaric acid N-hydroxysuccinimidate was added to 10 mg of hu-HEA125 antibody in 5 ml of PBS and reacted under slow rotation at 5° C. in the dark. After 16 h the solution was applied to a Sephadex G25 column (120×1.5 cm) equilibrated with PBS, and the protein fraction collected. Amanitin load was determined spectrophotometrically from the absorption difference at 310 nm of the protein solution against a blank containing the same concentration of the native antibody, using the molar extinction coefficient for amatoxins of 13.500 cm^{-1.}M⁻¹. Ratio α-amanitin: IgG of this preparation was ca. 8.

### 1.4 Binding Competition Analysis

Binding of amanitin-huHEA125 conjugate vs. non-conjugated huHEA125 antibody was analyzed in a competition experiment by flow cytometry. The α-amanitin-huHEA125 conjugate was synthesized as described above in sections 1.3.1 to 1.3.3.

Colo205 target cells (colon cancer metastasis) were washed twice in FACS buffer (Dulbecco's PBS with 1% heat-inactivated fetal calf serum and 0.1% sodium azide) counted and adjusted to 2×10⁷ cells per ml. Fifty μl of cell suspension was given to each well of a 96 well U-bottom microtiter plate to which 50 μl/well of FITC-labeled huHEA125 antibody was pipetted. Serial dilutions of amanitin-huHEA125 or huHEA125 ranging from 400 μg/ml to 10 ng/ml final dilution were added in triplicates in a volume of 50 μl/well and incubated for 1 h on ice. Subsequently, the plate was centrifuged (2 min at 2000 rpm) and the supernatant was removed from the cells. Cells were re-suspended in 150 μl of FACS buffer and centrifuged again. After two washing steps by centrifugation, cells were taken up in 100 μl/well of propidium iodide solution (1 μg/ml in FACS buffer) allowing discrimination of dead cells. Analysis was performed on a FACScan cytometer (Becton and Dickinson, Heidelberg, Germany) using CellQuest software.

As shown in FIG. 2 competition of binding to target cells with increasing amounts of huHEA125-amanitin conjugate or unmodified huHEA125 antibody revealed a comparable binding strength over the whole concentration range from 10 ng/ml to 400 μg/ml competing antibody or antibody conjugate. Therefore, the conjugation procedure did not significantly alter the affinity of huHEA125-amanitin to the target cells.

### Example 2

### Surface Expression of EpCAM Antigen on Various Carcinoma Cell Lines Detected by Indirect Immunofluorescence

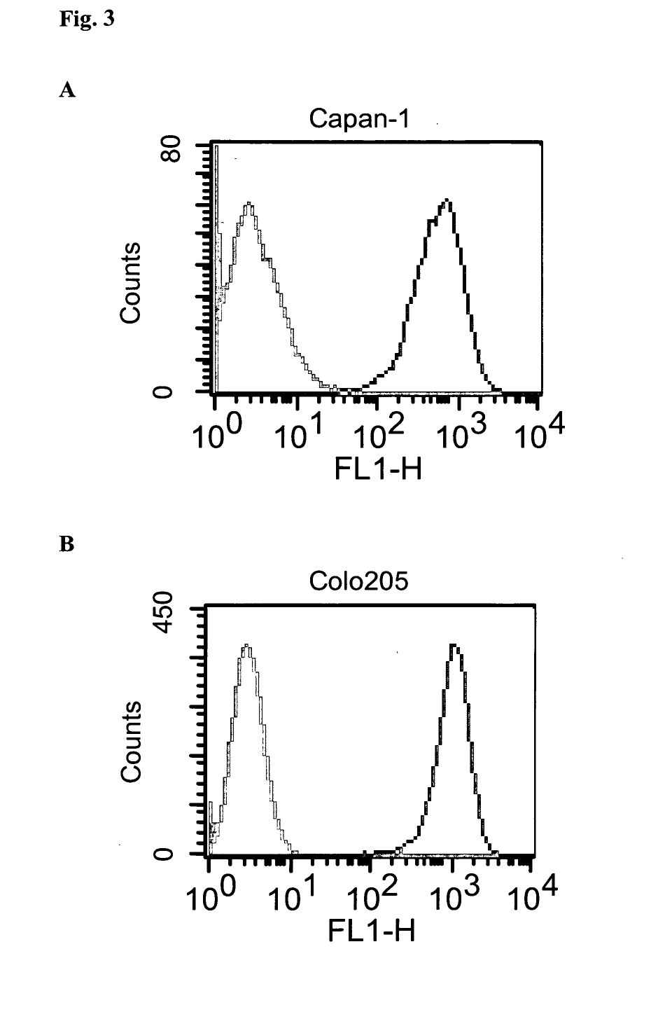

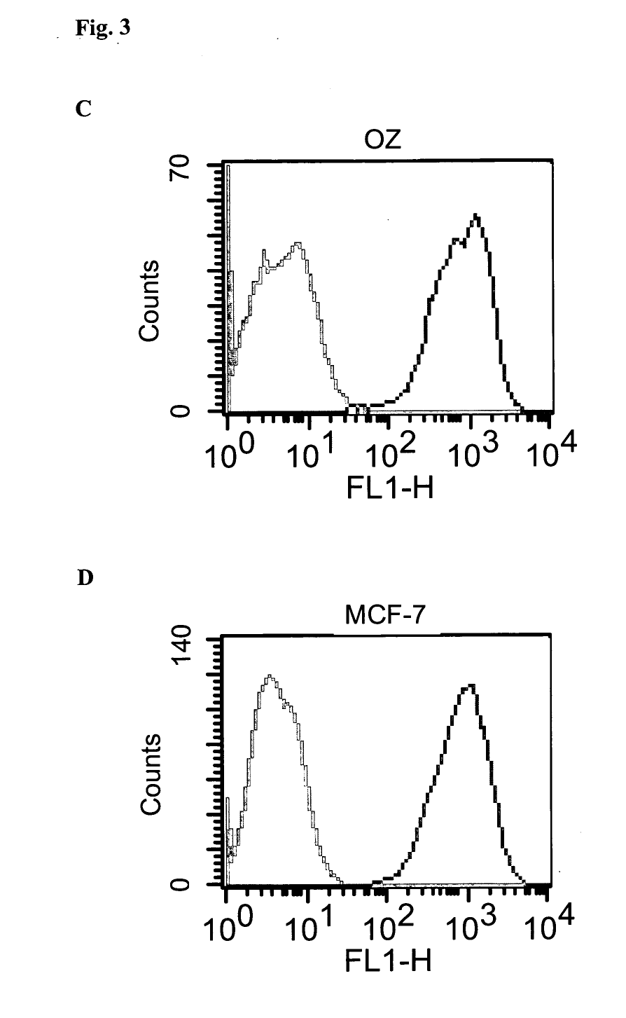

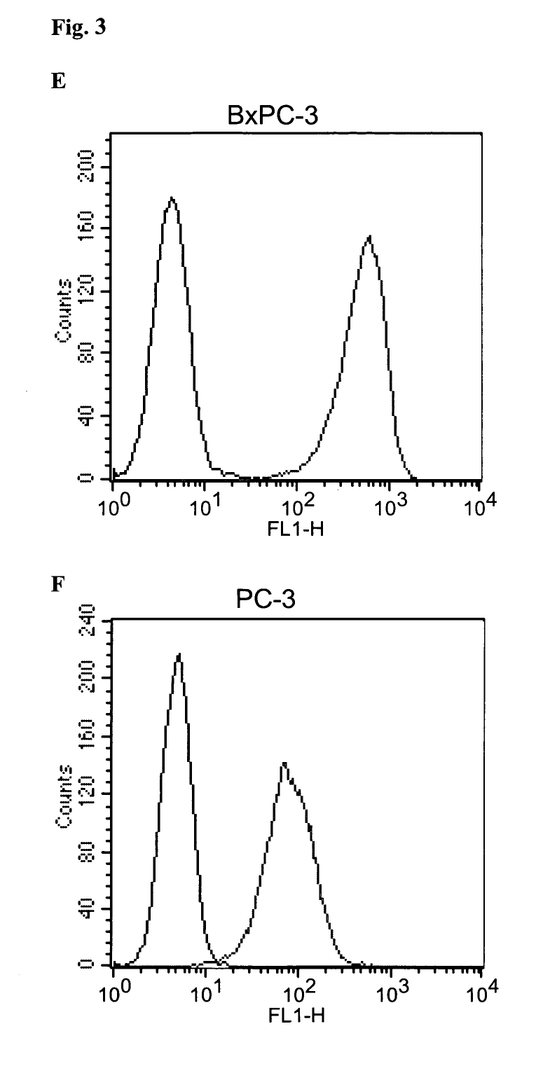

Cell lines Capan-1, Colo205, OZ, MCF-7, BxPC-3 and PC-3 were first incubated with either huHEA125 or Xolair®. After washing, binding of the primary antibody was visualized by FITC-labelled F(ab′)₂ goat anti-human IgG (H+L) as second step reagent. The results are shown in FIG. 3A (Capan-1), FIG. 3B (Colo205), FIG. 3C (OZ), FIG. 3D (MCF-7), FIG. 3E (BxPC-3), and FIG. 3F (PC-3). The grey-shaded histograms in the left side of each diagram show the results obtained with control antibody Xolair®; the histograms having a white area in the right side of each diagram show the results obtained with antibody huHEA125.

### Example 3

### Induction of Carcinoma Cell Proliferation Inhibition by Amanitin and Amanitin/Antibody Conjugates

### 3.1 Carcinoma Cell Lines

The following carcinoma cell lines were used for growth inhibition studies:

| | |
|---|---|
| Capan-1, BxPC-3 | human pancreatic adenocarcinoma |
| MCF-7 | human breast adenocarcinoma |
| Colo205 | human colon cancer metastasis |
| OZ | human cholangiocarcinoma |
| PC-3 | human prostate adenocarcinoma |

### 3.2 Proliferation Inhibition Assay

Inhibition of cell growth by amanitin-IgG conjugates was determined by incorporation of [³H]-thymidine. Serial dilutions of amanitin-huHEA125, amanitin-Xolair and free amanitin in complete medium (RPMI 1640 supplemented with 10% heat-inactivated FCS, 2 mM L-glutamine and 1 mM sodium pyruvate) ranging from 2×10⁻⁵ M to 6×10⁻¹³ were prepared in triplicates in a volume of 100 μl in the wells of a 96 well flat-bottom tissue culture microtiter plate. Cells were added in a volume of 50 μl per well at a density of 2×10⁴ per ml. Plates were incubated in a humidified atmosphere at 37° C. and 5% CO₂ for 72 or 96 h. At 20 h before the end of the assay, 1 μCi of [³H]-thymidine was added. Subsequently plates were processed with a Tomtec cell harvester and the incorporated radioactivity was determined by liquid scintillation counting (Wallac Betaplate Liquid Scintillation Counter, PerkinElmer Life and Analytical Sciences) and given as cpm.

In case of the pancreatic carcinoma cell line Capan-1 the huHEA125-amanitin immunotoxin induced growth arrest at amanitin concentrations of 1×10⁻¹¹ to 3×10⁻¹⁰ M as depicted in FIG. 4.

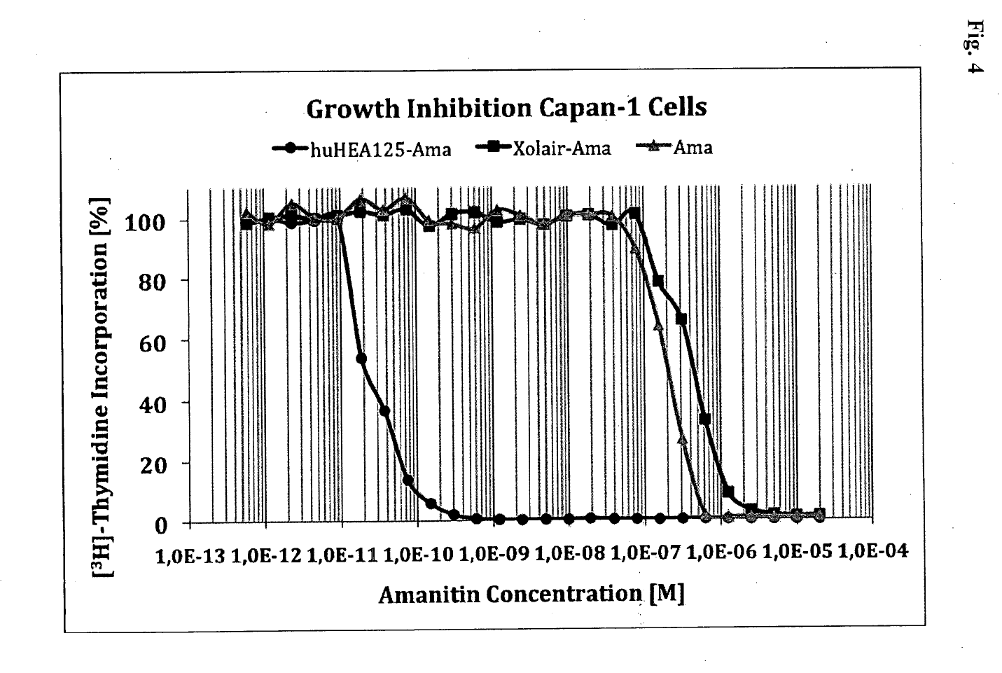

In case of the colon cancer cell line Colo205 the huHEA125-amanitin immunotoxin induced growth arrest at amanitin concentrations of 1×10⁻¹² to 4×10⁻¹¹ M as depicted in FIG. 5.

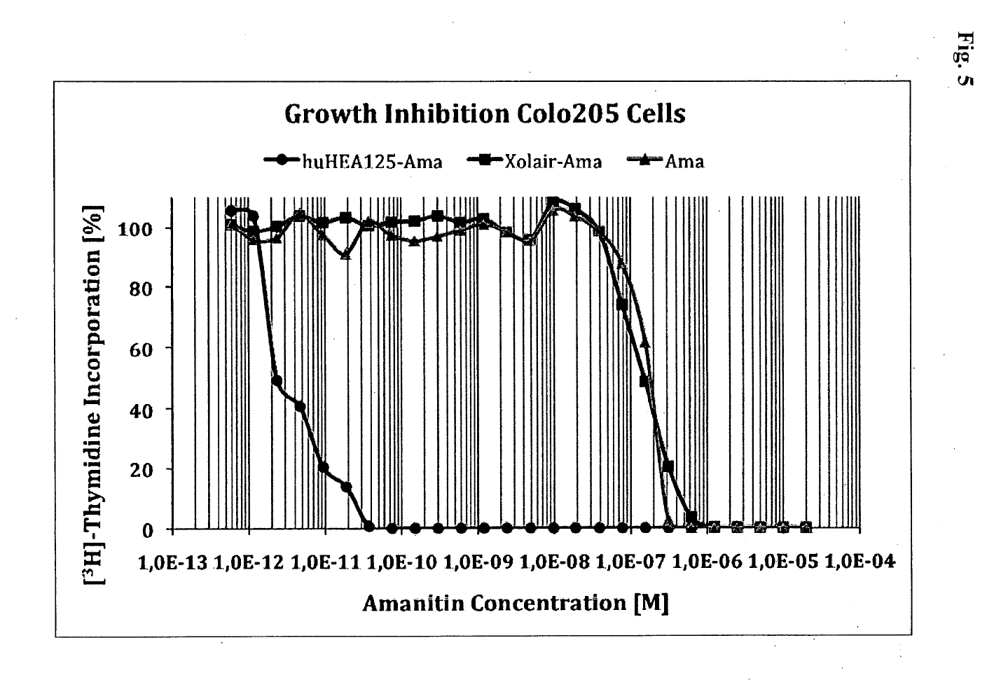

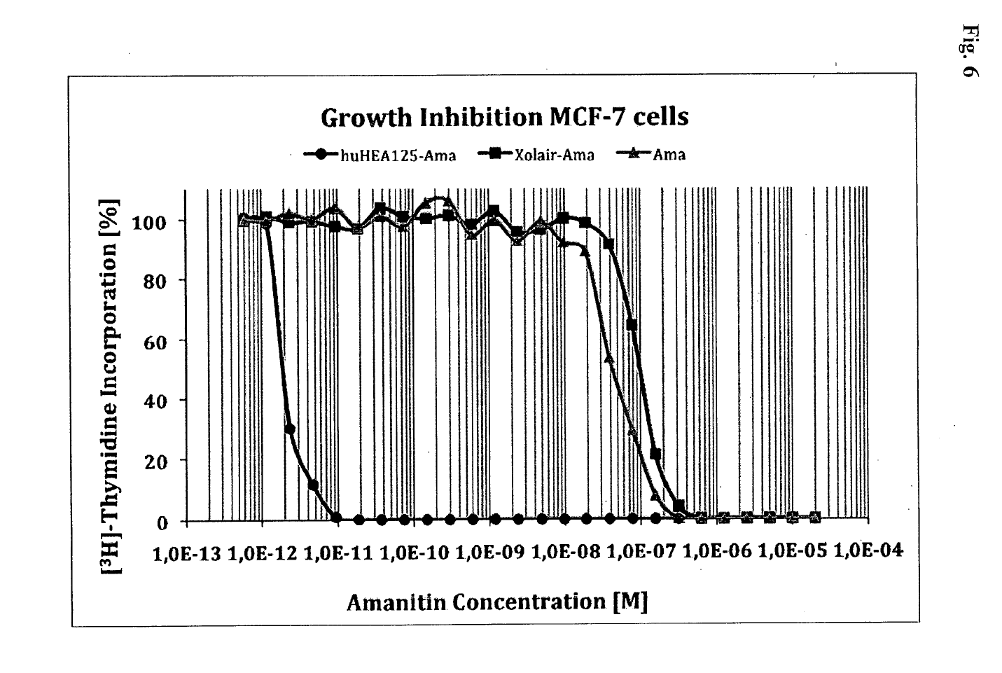

In case of the breast cancer cell line MCF-7 the huHEA125-amanitin immunotoxin induced growth arrest at amanitin concentrations of 1×10⁻¹² to 1×10⁻¹¹ M as depicted in FIG. 6.

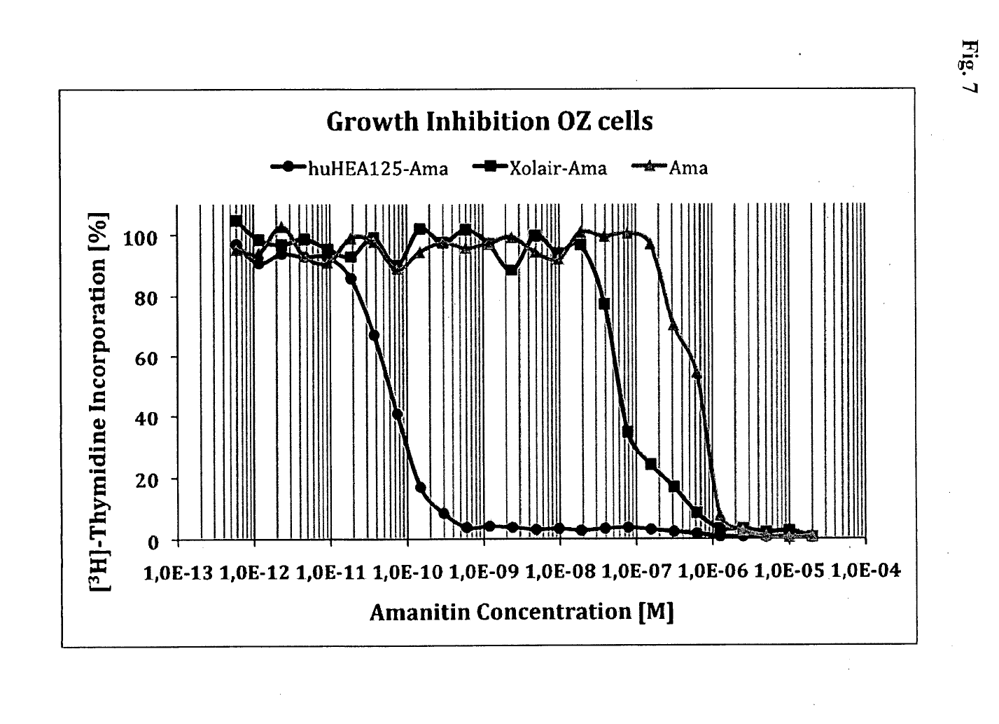

In case of the cholangiocarcinoma cell line OZ the huHEA125-amanitin immunotoxin induced growth arrest at amanitin concentrations of 1×10⁻¹¹ to 6×10⁻¹⁰ M as depicted in FIG. 7.

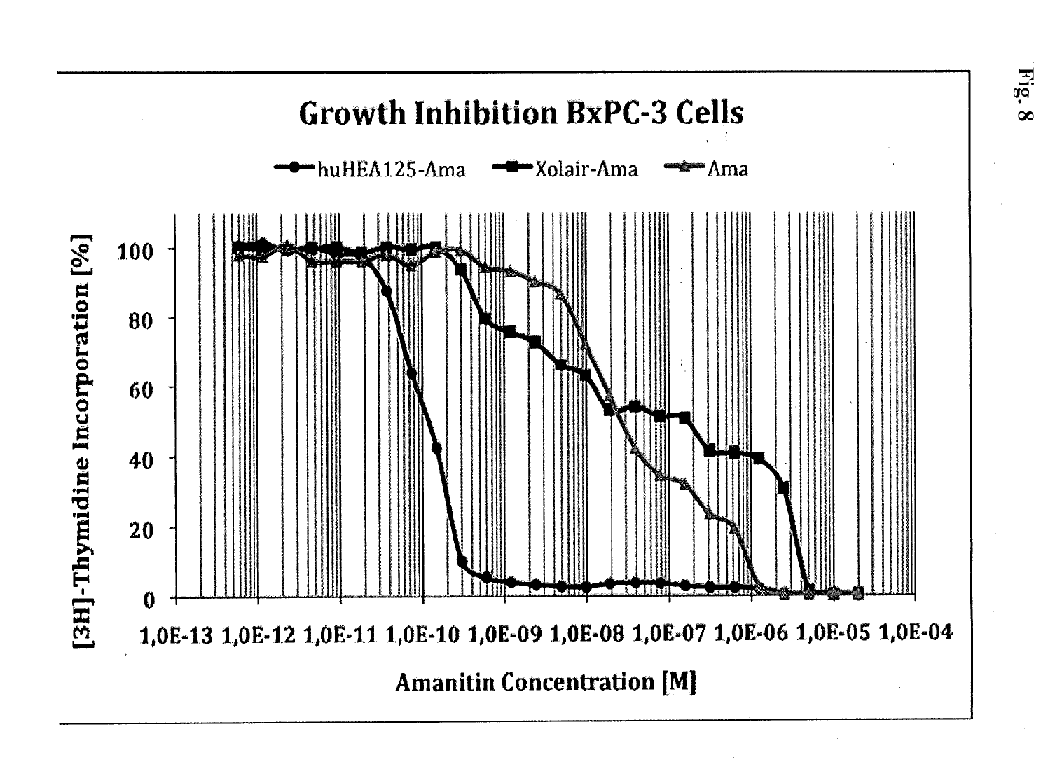

In case of the pancreatic cell line BxPC-3 the huHEA125-amanitin immunotoxin induced growth arrest at amanitin concentrations of 2×10⁻¹¹ to 6×10⁻¹⁰ M as depicted in FIG. 8.

### Example 4

### Inhibition of Tumor Growth in Vivo by Amanitin/Antibody Conjugate using Two Xenograft Mouse Tumor Models

Five- to six-week old immunodeficient NOD/SCID mice were used for all experiments. BxPC-3 pancreatic or PC-3 prostate tumor cells (5×10⁶ in 100 pi PBS) were transplanted subcutaneously to the right flank of the mice. Ten days later, when BxPC-3 tumors reached a volume of 30-80 mm³ and PC-3 tumors reached a volume of 40-190 mm³, the treatment was initiated. Animals received either control huHEA125 mAb at a dose of 15 mg/kg or huHEA125-amanitin conjugate (huHEA125-Ama) at a dose of 50 μg/kg of amanitin. Antibody and conjugate were administered as a single intraperitoneal injection.

Tumor growth was monitored for 16 days after initiation of the treatment. Tumor size was measured externally every third day using a caliper. Tumor volume was calculated according to the formula: V=π/6*a*b*c, where a, b and c are diameters in three dimensions. Data are presented as a relative tumor size/volume increase from the time of antibody administration.

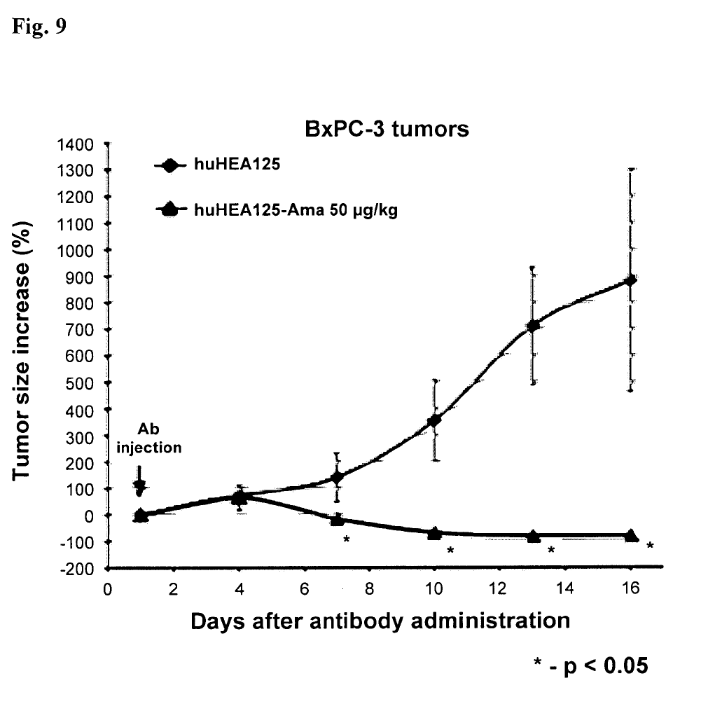

Administration of huHEA125-Ama at a dose of 50 μg/kg of amanitin was well tolerated by BxPC-3 tumor bearing mice (n=6). There was neither a decrease in body weight of the mice nor an elevation of liver enzymes (LDH, ALT, AST and AP was measured in the serum on the last day of experiment). The tumor growth was strongly inhibited by this dose of conjugate. All mice responded to treatment and tumor volume regressed dramatically starting from day 7 after the administration of conjugate. At the end of follow-up on day 16, tumor was completely eradicated in 50% of the mice. In contrast, in control mice that received non-conjugated huHEA125 mAb tumor volume increased by approximately 880% (FIG. 9).

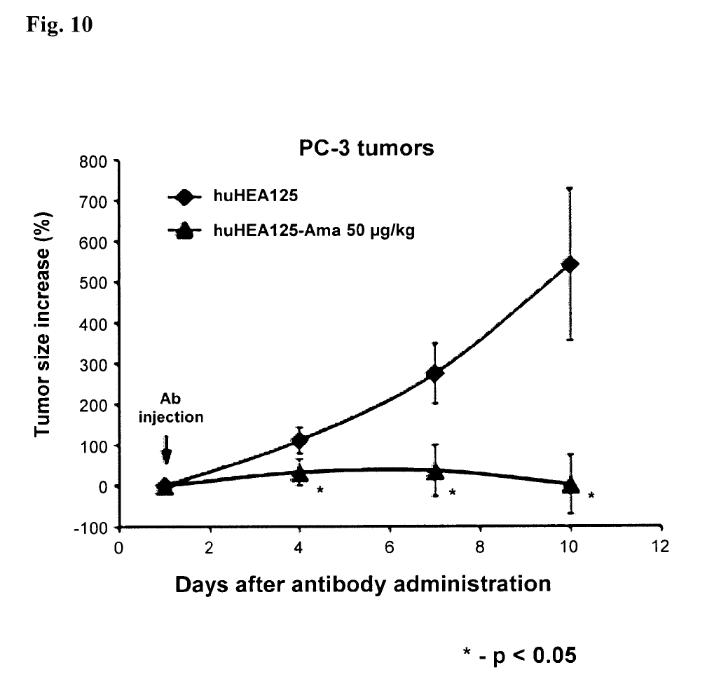

In case of PC-3 tumor bearing mice huHEA125-Ama at a dose of 50 μg/kg of amanitin was well tolerated. No decrease in the body weight of the mice was observed. The tumor growth was strongly retarded by this dose of the conjugate. Ten days after huHEA125-Ama administration, the tumor volume was similar to that at the initiation of the treatment. In contrast, in control mice that received non-conjugated huHEA125 mAb tumor volume increased by approximately 550%. The experiment was terminated on day 10 after treatment due to the large size of tumors in the control group (FIG. 10).

### REFERENCES

Al-Hajj M., Wicha M. S., Benito-Hernandez A., Morrison S. J., Clarke M. F. Prospective identification of tumorigenic breast cancer cells. Proc. Natl. Acad. Sci. USA 100(7), 3983-3988 (2003)
Allard W. J., Matera J., Miller M. C., Repollet M., Connelly M. C., Rao C., Tibbe A. G., Uhr J. W., Terstappen L. W. Tumor cells circulate in the peripheral blood of all major carcinomas but not in healthy subjects or patients with nonmalignant diseases. Clin. Cancer Res. 10(20), 6897-6904 (2004)
Baeuerle P. A. and Gires O. EpCAM (CD326) finding its role in cancer. Br. J. Cancer 96(3), 417-423 (2007)
Balzar M., Winter M. J., de Boer C. J., Litvinov S. V. The biology of the 17-1A antigen (Ep-CAM). J. Mol. Med. 77(10), 699-712 (1999)
Binz H. K., Amstutz P., Plückthun A. Engineering novel binding proteins from nonimmunoglobulin domains. Nat Biotechnol. 23(10):1257-1268 (2005)
Brody E. N. and Gold L., Aptamers as therapeutic and diagnostic agents. J. Biotechnol. 74(1):5-13 (2000)
Dalerba P., Dylla S. J., Park I. K., Liu R., Wang X., Cho R. W., Hoey T., Gurney A., Huang E. H., Simeone D. M., Shelton A. A., Parmiani G., Castelli C., Clarke M. F. Phenotypic characterization of human colorectal cancer stem cells. Proc. Natl. Acad. Sci. USA 104(24), 10158-10163 (2007)
Gastl G., Spizzo G., Obrist P., Dünser M., Mikuz G. Ep-CAM overexpression in breast cancer as a predictor of survival. Lancet 356(9246), 1981-1982 (2000)
Holliger P., Prospero T., Winter G. “Diabodies”: small bivalent and bispecific antibody fragments. Proc. Natl. Acad. Sci. U.S.A. 90(14), 6444-6448 (1993)
Leuenberger, H. G. W, Nagel, B. and Kölbl, H. eds. “A multilingual glossary of biotechnological terms: (IUPAC Recommendations)”, Helvetica Chimica Acta, CH-4010 Basel, Switzerland), 1995
Li C., Heidt D. G., Dalerba P., Burant C. F., Zhang L., Adsay V., Wicha M., Clarke M. F., Simeone D. M. Identification of pancreatic cancer stem cells. Cancer Res. 67(3), 1030-1037 (2007)
Moldenhauer G., Momburg F., Moller P., Schwartz R., Hämmerling G. J. Epithelium-specific surface glycoprotein of Mr 34,000 is a widely distributed human carcinoma marker. Br. J. Cancer 56(6), 714-721 (1987)
Momburg F., Moldenhauer G., Hämmerling G. J., Möller P. Immunohistochemical study of the expression of a Mr 34,000 human epithelium-specific surface glycoprotein in normal and malignant tissues. Cancer Res. 47(11), 2883-2891 (1987)
Nagrath S., Sequist L. V., Maheswaran S., Bell D. W., Irimia D., Ulkus L., Smith M. R., Kwak E. L., Digumarthy S., Muzikansky A., Ryan P., Balis U. J., Tompkins R. G., Haber D. A., Toner M. Isolation of rare circulating tumour cells in cancer patients by microchip technology. Nature 450(7173), 1235-1239 (2007)
Spizzo G., Obrist P., Ensinger C., Theurl I., Dünser M., Ramoni A., Gunsilius E., Eibl G., Mikuz G., Gastl G. Prognostic significance of Ep-CAM AND Her-2/neu overexpression in invasive breast cancer. Int. J. Cancer 98(6), 883-888 (2002)
Spizzo G., Went P., Dimhofer S., Obrist P., Simon R., Spichtin H., Maurer R., Metzger U., von Castelberg B., Bart R., Stopatschinskaya S., Köchli O. R., Haas P., Mross F., Zuber M., Dietrich H., Bischoff S., Mirlacher M., Sauter G., Gastl G. High Ep-CAM expression is associated with poor prognosis in node-positive breast cancer. Breast Cancer Res. Treat. 86(3), 207-213 (2004)
Trzpis M., McLaughlin P. M., de Leij L. M., Harmsen M. C. Epithelial cell adhesion molecule: more than a carcinoma marker and adhesion molecule. Am. J. Pathol. 171(2), 386-395 (2007)
Varga M., Obrist P., Schneeberger S., Mühlmann G., Felgel-Farnholz C., Fong D., Zitt M., Brunhuber T., Schäfer G., Gastl G., Spizzo G. Overexpression of epithelial cell adhesion molecule antigen in gallbladder carcinoma is an independent marker for poor survival. Clin. Cancer Res. 10(9), 3131-3136 (2004)
Went P. T., Lugli A., Meier S., Bundi M., Mirlacher M., Sauter G., Dirnhofer S. Frequent EpCam protein expression in human carcinomas. Hum. Pathol. 35(1), 122-128, 2004
Wieland, T. and Faulstich H. Amatoxins, phallotoxins, phallolysin, and antamanide: the biologically active components of poisonous Amanita mushrooms. CRC Crit. Rev. Biochem. 5(3), 185-260 (1978)
Winter M. J., Nagtegaal I. D., van Krieken J. H., Litvinov S. V. The epithelial cell adhesion molecule (Ep-CAM) as a morphoregulatory molecule is a tool in surgical pathology. Am. J. Pathol. 163(6), 2139-2148 (2003)

1. An antibody toxin conjugate for the treatment of pancreatic cancer, cholangiocarcinoma, or colorectal cancer in a patient, wherein the conjugate comprises
(i) an antibody or antigen binding fragment thereof specifically binding to an epitope of epithelial cell adhesion molecule (EpCAM);
(ii) an amatoxin; and
(iii) optionally a linker L1.

2. The conjugate of claim 1 wherein the antibody or antigen binding fragment thereof is selected from a diabody, a tetrabody, a nanobody, a chimeric antibody, a deimmunized antibody, a humanized antibody or a human antibody.

3. The conjugate of claim 1 or 2 wherein the antigen binding fragment is selected from the group consisting of Fab, F(ab′)₂, Fd, Fv, single-chain Fv, and disulfide-linked Fvs (dsFv).

4. The conjugate of any one of claims 1 to 3 wherein the epitope of EpCAM is an epitope of human EpCAM.

5. The conjugate of any one of claims 1 to 4 wherein the antibody or the antigen binding fragment thereof comprises
(a) the CDR3 domain (SEQ ID NO: 22) of the heavy chain of huHEA125; and/or
(b) the CDR3 domain (SEQ ID NO: 25) of the light chain of huHEA125.

6. The conjugate of claim 5 wherein the antibody or the antigen binding fragment thereof additionally comprises one or more of the following:
(a) the CDR2 domain (SEQ ID NO: 21) of the heavy chain of huHEA125;
(b) the CDR1 domain (SEQ ID NO: 20) of the heavy chain of huHEA125;
(c) the CDR2 domain (SEQ ID NO: 24) of the light chain of huHEA125; and
(d) the CDR1 domain (SEQ ID NO: 23) of the light chain of huHEA125.

7. The conjugate of any one of claims 1 to 6 wherein the antibody or the antigen binding fragment thereof comprises the VH domain of huHEA125 (SEQ ID NO: 3) and/or the VL domain of huHEA125 (SEQ ID NO: 12).

8. The conjugate of any one of claims 1 to 7 wherein the antibody or the antigen binding fragment thereof comprises
(a) either the membrane-bound form of the heavy chain of huHEA125 (SEQ ID NO: 1) or the soluble form of the heavy chain of huHEA125 (SEQ ID NO: 2); and/or
(b) the light chain of huHEA125 (SEQ ID NO: 11).

9. The conjugate of any one of claims 1 to 8 wherein the amatoxin is selected from α-amanitin, β-amanitin, γ-amanitin, £-amanitin, amanin, amaninamide, amanullin, or amanullinic acid, or salts or analogs thereof.

10. An antibody toxin conjugate comprising
(i) an antibody or an antigen binding fragment thereof specifically binding to epithelial cell adhesion molecule (EpCAM), wherein the antibody or an antigen binding fragment thereof comprises:
(a) the heavy chain of huHEA125, wherein the heavy chain is selected from the group consisting of:
(a1) the membrane-bound form of the heavy chain according to SEQ ID NO: 1,
wherein the variable domain of the heavy chain VH as shown in SEQ ID NO: 3 comprises between 0 and 10 amino acid exchanges, between 0 and 10 amino acid deletions and/or between 0 and 10 amino acid additions positioned in the framework regions of VH, and
wherein the constant domain of the heavy chain as shown in SEQ ID NO: 26 comprises between 0 and 10 amino acid exchanges, between 0 and 10 amino acid deletions and/or between 0 and 10 amino acid additions; and
(a2) the soluble form of the heavy chain according to SEQ ID NO: 2,
wherein the variable domain of the heavy chain VH as shown in SEQ ID NO: 3 comprises between 0 and 10 amino acid exchanges, between 0 and 10 amino acid deletions and/or between 0 and 10 amino acid additions positioned in the framework regions of VH, and
wherein the constant domain of the heavy chain as shown in SEQ ID NO: 27 comprises between 0 and 10 amino acid exchanges, between 0 and 10 amino acid deletions and/or between 0 and 10 amino acid additions;
and
(b) the light chain of huHEA125 according to SEQ ID NO: 11, wherein the variable domain of the light chain VL as shown in SEQ ID NO: 12 comprises between 0 and 10 amino acid exchanges, between 0 and 10 amino acid deletions and/or between 0 and 10 amino acid additions positioned in the framework regions of VL, and wherein the constant domain of the light chain CL as shown in SEQ ID NO: 28 comprises between 0 and 10 amino acid exchanges, between 0 and 10 amino acid deletions and/or between 0 and 10 amino acid additions.
(ii) an amatoxin; and
(iii) optionally a linker L2.

11. The conjugate of claim 10 wherein the antibody or antigen binding fragment thereof is selected from a chimeric antibody, a deimmunized antibody, a humanized antibody or a human antibody.

12. The conjugate of claim 10 or 11 wherein the antigen binding fragment is selected from the group consisting of Fab, F(ab′)₂, and Fd.

13. The conjugate of any one of claims 10 to 12 wherein the antibody is huHEA125 or an antigen binding fragment thereof.

14. The conjugate of any one of claims 10 to 13 wherein the amatoxin is selected from α-amanitin, β-amanitin, γ-amanitin, £-amanitin, amanin, amaninamide, amanullin, or amanullinic acid, or salts or analogs thereof.

15. The conjugate of any one of claims 10 to 14 for use in medicine.

16. The conjugate of any one of claims 10 to 14 for the treatment of cancer in a patient, wherein the cancer is selected from the group consisting of pancreatic cancer, cholangiocarcinoma, breast cancer and colorectal cancer.

17. A target-binding moiety toxin conjugate comprising:
(i) a target-binding moiety specifically binding to an epitope of epithelial cell adhesion molecule (EpCAM)
(ii) an amatoxin; and
(iii) optionally a linker L3;
wherein the amatoxin is connected to the target-binding moiety or, if present, to the linker L3 via the δC-atom of amatoxin amino acid 3.

18. The target-binding moiety toxin conjugate of claim 17, wherein the amatoxin is connected to the target-binding moiety or, if present, to the linker L3 via an oxygen atom bound to the δC-atom of amatoxin amino acid 3.

19. The target-binding moiety toxin conjugate of claim 17 or 18, wherein the amatoxin is connected to the target-binding moiety or, if present, to the linker L3 via an ester linkage, an ether linkage or a urethane linkage.

20. The target-binding moiety toxin conjugate of any one of claims 17 to 19, wherein the linker L3 is present and the conjugate has one of the following structures:
(i) amatoxin-δC—O—C(O)-L3-C(O)-NH-target-binding moiety;
(ii) amatoxin-δC—O-L3-C(O)—NH-target-binding moiety; or
(iii) amatoxin-δC—O—C(O)—NH-L3-C(O)—NH-target-binding moiety.

21. The target-binding moiety toxin conjugate of any one of claims 17 to 20, wherein the target-binding moiety is connected to the amatoxin or, if present, to the linker L3 via an amino group present in the target-binding moiety.

22. The target-binding moiety toxin conjugate of any one of claims 17 to 21, wherein the amatoxin is selected from α-amanitin, β-amanitin, γ-amanitin, £-amanitin, amanin, amaninamide, amanullin, or amanullinic acid, or from salts or analogs thereof.

23. The target-binding moiety toxin conjugate of any one of claims 17 to 22, wherein the linker L3 is an alkyl, heteroalkyl, alkenyl, heteroalkenyl, alkynyl, heteroalkynyl, cycloalkyl, heterocycloalkyl, aryl, heteroaryl, aralkyl, or a heteroaralkyl group, optionally substituted.

24. The target-binding moiety toxin conjugate of any one of claims 17 to 23, wherein the linker L3 comprises a disulfide bond.

25. The target-binding moiety toxin conjugate of any one of claims 17 to 24 wherein the target-binding moiety specifically binds to an epitope that is present on a tumour cell.

26. The target-binding moiety toxin conjugate of any one of claims 17 to 25, wherein the target binding moiety is selected from the group consisting of:
antibody or antigen-binding fragment thereof;
(ii) antibody-like protein; and
(iii) nucleic acid aptamer.

27. The target-binding moiety toxin conjugate of claim 26, wherein the antibody or the antigen-binding fragment thereof is selected from a diabody, a tetrabody, a nanobody, a chimeric antibody, a deimmunized antibody, a humanized antibody or a human antibody.

28. The target-binding moiety toxin conjugate of claim 26 or 27, wherein the antigen binding fragment is selected from the group consisting of Fab, F(ab′)₂, Fd, Fv, single-chain Fv, and disulfide-linked Fvs (dsFv).

29. The target-binding moiety toxin conjugate of claims 26 to 27 wherein the antibody or the antigen binding fragment thereof comprises
(a) either the membrane-bound form of the heavy chain of huHEA125 (SEQ ID NO: 1) or the soluble form of the heavy chain of huHEA125 (SEQ ID NO: 2); and/or
(b) the light chain of huHEA125 (SEQ ID NO: 11).

30. The target-binding moiety toxin conjugate of any one of claims 17 to 29 for use in medicine.

31. The target-binding moiety toxin conjugate of any one of claims 17 to 30 for the treatment of cancer in a patient, wherein the cancer is selected from the group consisting of pancreatic cancer, cholangiocarcinoma, breast cancer, colorectal cancer, lung cancer, prostate cancer, ovarian cancer, stomach cancer, kidney cancer, malignant melanoma, leukemia and malignant lymphoma.

32. Pharmaceutical composition comprising the antibody toxin conjugate according to any one of claims 1 to 14 or the target-binding moiety toxin conjugate according to any one of claims 17 to 29 and further comprising one or more pharmaceutically acceptable diluents, carriers, excipients, fillers, binders, lubricants, glidants, disintegrants, adsorbents; and/or preservatives.
